(19) United States
(12) Patent Application Publication
(10) Pub. No.: US 2026/0072185 A1
(43) Pub. Date: Mar. 12, 2026

(54) RADIATION IMAGING APPARATUS, WIRELESS POWER FEEDING APPARATUS, AND RADIATION IMAGING SYSTEM

(71) Applicant: CANON KABUSHIKI KAISHA, Tokyo (JP)

(72) Inventors: TAKASHI FUKUSHIMA, Kanagawa (JP); YURI YOSHIMURA, Tokyo (JP); HIROSHI SASAKI, Kanagawa (JP)

(21) Appl. No.: 19/388,529

(22) Filed: Nov. 13, 2025

Related U.S. Application Data
(63) Continuation of application No. PCT/JP2024/020801, filed on Jun. 7, 2024.

(30) Foreign Application Priority Data
Jun. 13, 2023 (JP) 2023-097148
Nov. 8, 2023 (JP) 2023-191072
Mar. 27, 2024 (JP) 2024-051814
May 14, 2024 (JP) 2024-078892

(51) Int. Cl. G01T 1/20; A61B 6/00; G01K 3/00; H02J 50/10; H02J 50/70; H02J 50/80; H02J 50/90

(52) U.S. Cl. CPC G01T 1/20188; A61B 6/4405; A61B 6/4411; A61B 6/56; G01K 3/005; H02J 50/10; H02J 50/70; H02J 50/80; H02J 50/90

(57) ABSTRACT

A radiation imaging apparatus includes a radiation detector configured to convert incident radiation into an electric signal regarding a radiation image, at least one power reception unit configured to receive power from outside, and a housing containing the radiation detector and the power reception unit, the housing includes a thick portion and a thin portion integrated together in an internal space, and a panel portion of the radiation detector and the power reception unit are placed in the thin portion.

### CROSS-REFERENCE TO RELATED APPLICATIONS

This application is a Continuation of International Patent Application No. PCT/JP2024/020801, filed Jun. 7, 2024, which claims the benefit of Japanese Patent Applications No. 2023-097148, filed Jun. 13, 2023, No. 2023-191072, filed Nov. 8, 2023, No. 2024-051814, filed Mar. 27, 2024, and No. 2024-078892, filed May 14, 2024, all of which are hereby incorporated by reference herein in their entirety.

### BACKGROUND

### Field of the Technology

The present disclosure relates to a radiation imaging apparatus, a wireless power feeding apparatus, and a radiation imaging system.

### Description of the Related Art

In the medical diagnosis field, a radiation imaging apparatus that obtains a radiation image by detecting the intensity distribution of radiation transmitted through a target object is widely used.

To enable rapid and wide-area imaging of anatomical regions, there is a demand for a radiation imaging apparatus that is thin, easy to handle, and highly operable. To address such an issue, Japanese Patent No. 7031014 describes a configuration in which the thickness of a radiation detector of a radiation imaging apparatus is thinned. For example, to charge a battery built into a radiation imaging apparatus, a power reception unit such as a connection connector with an external power supply or the like is often disposed in a radiation imaging apparatus. Japanese Patent No. 5638372 describes a configuration in which the thickness of a region outside a radiation detector is thickened, a power reception unit of a connector is disposed in this thickened portion, and a thick portion and a thin portion as separate portions are attachable to and detachable from each other.

In a case where a power reception unit is provided in a region outside a radiation detector, a radiation imaging apparatus has a size different from that of a conventional radiation imaging apparatus. Particularly, there is a possibility that the thickness of the region outside the radiation detector increases compared to the thickness the conventional radiation imaging apparatus. In this case, there is a possibility that the radiation imaging apparatus cannot be accommodated in an accommodation box of a medical cart designed on the premise of the thickness of the conventional radiation imaging apparatus. There is an issue where a charging plug built into the accommodation box of the medical cart or the like and a connector provided in the radiation imaging apparatus do not fit each other.

### SUMMARY

In view of the above issue, the present disclosure is directed to providing a radiation imaging apparatus that achieves a suitable configuration and suitable placement of a power reception unit that suits a case where the thickness of a radiation detector is thinned.

According to an aspect of the present disclosure, a radiation imaging apparatus includes a radiation detector configured to detect radiation, a power supply unit configured to supply power to the radiation detector, a housing configured to accommodate the radiation detector in a thin portion and accommodate the power supply unit in a thick portion, and a power reception unit disposed in the thin portion and configured to receive supply of power from outside.

According to another aspect of the present disclosure, a radiation imaging apparatus includes a radiation detector configured to detect radiation, a power supply unit configured to supply power to the radiation detector, a housing configured to accommodate the radiation detector in a thin portion and accommodate the power supply unit in a thick portion, and a power reception unit disposed in the thick portion and configured to receive supply of power from outside, wherein the power reception unit is any of a sloping surface power reception unit disposed in proximity of a sloping surface linking the thick portion and the thin portion, a side surface power reception unit disposed on a side surface that does not come into contact with a flat floor surface among side surfaces of the thick portion, and a wireless power reception unit configured to receive wireless feeding of power.

According to yet another aspect of the present disclosure, a wireless power feeding apparatus that wirelessly feeds power to a radiation imaging apparatus including a wireless power reception unit for receiving wireless feeding of power, the wireless power feeding apparatus includes an accommodation unit configured to accommodate the radiation imaging apparatus, a wireless power transmission unit configured to wirelessly feed power to the wireless power reception unit, and an adjustment mechanism configured to passively operate in a case where the radiation imaging apparatus is accommodated in the accommodation unit, and adjust a position of the radiation imaging apparatus in such a manner that the wireless power transmission unit and the wireless power reception unit have a positional relationship where the wireless power feeding apparatus is able to wirelessly feed power to the radiation imaging apparatus.

Features of the present disclosure will become apparent from the following description of embodiments with reference to the attached drawings.

### BRIEF DESCRIPTION OF THE DRAWINGS

FIG. 1 is a perspective view illustrating a radiation imaging apparatus according to a first embodiment.

FIG. 2 is a partial cross-sectional view along a dashed line B-B′ in FIG. 1.

FIG. 3 is a partial cross-sectional view of a radiation imaging apparatus according to a first variation of the first embodiment similar to that in FIG. 2.

FIG. 4 is a partial cross-sectional view of a radiation imaging apparatus according to a second variation of the first embodiment similar to that in FIG. 2.

FIG. 5 is a partial cross-sectional view of a radiation imaging apparatus according to a third variation of the first embodiment similar to that in FIG. 2.

FIG. 6 is a partial cross-sectional view of a radiation imaging apparatus according to a fourth variation of the first embodiment similar to that in FIG. 2.

FIG. 7 is a perspective view illustrating a radiation imaging apparatus according to a second embodiment.

FIG. 8 is a block diagram illustrating a configuration of the radiation imaging apparatus according to the second embodiment.

FIG. 9 is a perspective view illustrating a radiation imaging apparatus according to a third embodiment.

FIG. 10A is a partial cross-sectional view of the radiation imaging apparatus according to the third embodiment similar to that in FIG. 2.

FIG. 10B is a partial cross-sectional view of the radiation imaging apparatus according to the third embodiment similar to that in FIG. 2.

FIG. 11 is a partial cross-sectional view of a radiation imaging apparatus according to a fourth embodiment similar to that in FIG. 2.

FIG. 12 is a block diagram illustrating a configuration of the radiation imaging apparatus according to the fourth embodiment.

FIG. 13 is a flowchart illustrating an example of control according to temperature according to the fourth embodiment.

FIG. 14 is a block diagram illustrating a configuration of a radiation imaging apparatus according to a fifth embodiment.

FIG. 15 is a flowchart illustrating an example of control according to time according to the fifth embodiment.

FIG. 16 is a schematic view illustrating a radiation imaging system.

FIG. 17A is a perspective view illustrating a radiation imaging apparatus according to a sixth embodiment.

FIG. 17B is a perspective view illustrating the radiation imaging apparatus according to the sixth embodiment.

FIG. 18 is a cross-sectional view along a dashed-dotted line A-A′ in FIG. 17A.

FIG. 19 is a plan view of a back surface side illustrating an internal configuration of the radiation imaging apparatus according to the sixth embodiment.

FIG. 20 is a side view illustrating a state where the radiation imaging apparatus according to the sixth embodiment falls.

FIG. 21 is a perspective view illustrating a medical cart including an accommodation box for the radiation imaging apparatus according to the sixth embodiment.

FIG. 22A is a cross-sectional view of the accommodation box along a dashed-dotted line B-B′ in FIG. 21.

FIG. 22B is a cross-sectional view of the accommodation box along the dashed-dotted line B-B′ in FIG. 21.

FIG. 22C is a cross-sectional view of the accommodation box along the dashed-dotted line B-B′ in FIG. 21.

FIG. 23 is a side view illustrating another example of the medical cart including the accommodation box for the radiation imaging apparatus according to the sixth embodiment.

FIG. 24 is a cross-sectional view illustrating a radiation imaging apparatus according to a variation of the sixth embodiment.

FIG. 25 is a cross-sectional view illustrating a medical cart including an accommodation box for the radiation imaging apparatus according to the variation of the sixth embodiment.

FIG. 26 is a schematic view illustrating a radiation imaging apparatus according to a seventh embodiment.

FIG. 27A is an enlarged schematic view of a peripheral portion of a power reception connector of the radiation imaging apparatus according to the seventh embodiment.

FIG. 27B is an enlarged schematic view of the peripheral portion of the power reception connector of the radiation imaging apparatus according to the seventh embodiment.

FIG. 28 is a side view illustrating a state where the radiation imaging apparatus according to the seventh embodiment falls.

FIG. 29 is a perspective view illustrating a medical cart including an accommodation box for the radiation imaging apparatus according to the seventh embodiment.

FIGS. 30A and 30B are partial cutaway side views illustrating a portion of the accommodation box for the radiation imaging apparatus according to the seventh embodiment when viewed in a direction S in FIG. 29.

FIG. 31A is a schematic view illustrating the accommodation box according to the seventh embodiment.

FIG. 31B is a schematic view illustrating the accommodation box according to the seventh embodiment.

FIG. 31C is a schematic view illustrating the accommodation box according to the seventh embodiment.

FIG. 31D is a schematic view illustrating the accommodation box according to the seventh embodiment.

FIG. 32 is a schematic view illustrating a radiation imaging apparatus according to an eighth embodiment.

FIG. 33 is a cross-sectional view along a dashed-dotted line D-D′ in FIG. 32.

FIG. 34 is a side view illustrating a state where the radiation imaging apparatus according to the eighth embodiment falls.

FIG. 35 is a top perspective view of a cartridge according to a ninth embodiment.

FIG. 36 is a bottom perspective view of the cartridge according to the ninth embodiment.

FIG. 37 is a cross-sectional view along a direction of a dashed-dotted line B-B′ in FIG. 35.

FIG. 38 is a top perspective view of a cartridge according to the ninth embodiment.

FIG. 39 is a bottom perspective view of the cartridge according to the ninth embodiment.

FIG. 40 is a cross-sectional view along a direction of a dashed-dotted line C-C′ in FIG. 38.

FIG. 41 is a perspective view of a charging apparatus according to the ninth embodiment.

FIG. 42 is a top view of the charging apparatus according to the ninth embodiment.

FIG. 43A is a perspective view illustrating a series of behaviors when a cartridge is accommodated in the charging apparatus according to the ninth embodiment.

FIG. 43B is a perspective view illustrating the series of behaviors when the cartridge is accommodated in the charging apparatus according to the ninth embodiment.

FIG. 43C is a perspective view illustrating the series of behaviors when the cartridge is accommodated in the charging apparatus according to the ninth embodiment.

FIG. 44 is a perspective view illustrating a state where a cartridge is accommodated in the charging apparatus according to the ninth embodiment.

FIG. 45 is a perspective view of a charging apparatus according to a tenth embodiment.

FIG. 46 is a side view of the charging apparatus according to the tenth embodiment.

FIG. 47A is a perspective view illustrating a series of behaviors when a cartridge is accommodated in the charging apparatus according to the tenth embodiment.

FIG. 47B is a perspective view illustrating the series of behaviors when the cartridge is accommodated in the charging apparatus according to the tenth embodiment.

FIG. 47C is a perspective view illustrating the series of behaviors when the cartridge is accommodated in the charging apparatus according to the tenth embodiment.

FIG. 48 is a front view illustrating a state where a cartridge having the smallest height among cartridges intended to be accommodated in the charging apparatus according to the tenth embodiment is accommodated.

### DESCRIPTION OF THE EMBODIMENTS

Hereinafter, preferred embodiments to which the present disclosure can be applied will be described in detail with reference to the drawings. In the following description, components that are common across a plurality of drawings are denoted by the same reference numerals. Therefore, common components may be described by referring to the plurality of drawings, and redundant explanations for components with the same reference numerals may be omitted as appropriate. The radiation in the embodiments may include beams formed by particles (including photons) emitted through radioactive decay, such as alpha rays, beta rays, and gamma rays. In addition, beams with energy equal to or greater than these, such as X-rays, particle beams, and cosmic rays, may also be included.

### First Embodiment

With reference to the drawings, a first embodiment is described in detail below.

### [Radiation Imaging Apparatus According to Present Embodiment]

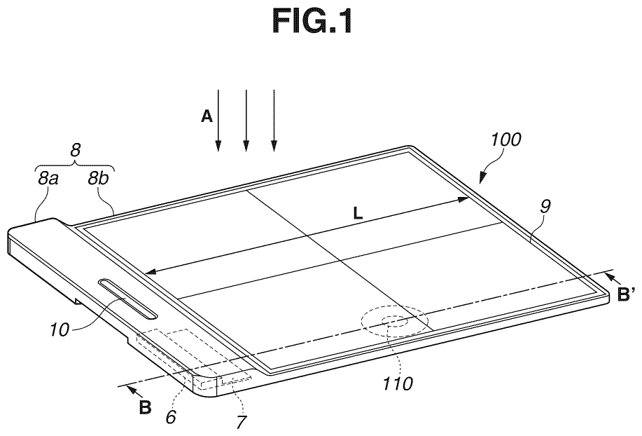

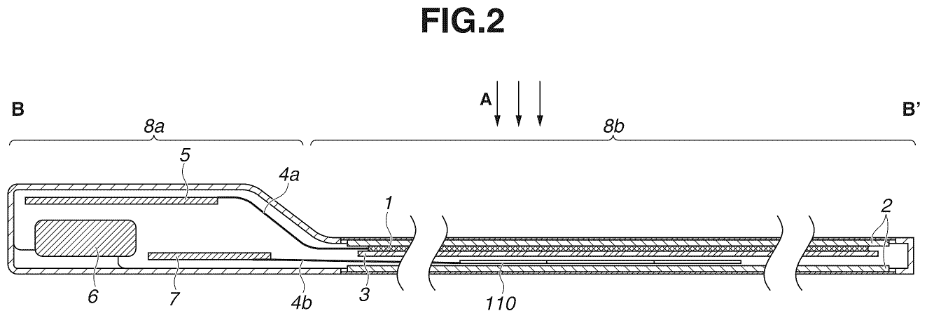

FIG. 1 is a perspective view illustrating a radiation imaging apparatus according to the present embodiment. FIG. 2 is a partial cross-sectional view along a dashed line B-B′ in FIG. 1.

A radiation imaging apparatus 100 detects radiation emitted from a radiation generating apparatus (not illustrated) and transmitted through an object, using a radiation detection panel 1. Information regarding an image acquired by the radiation imaging apparatus 100 is transferred to the outside, displayed on a predetermined display monitor or the like, and used for a diagnosis or the like.

The radiation detection panel 1 is a radiation detection panel using a so-called indirect conversion method including a sensor substrate on which many photoelectric conversion elements (sensors) are disposed on an upper portion of the sensor substrate, a phosphor layer (a scintillator layer) disposed on the sensor substrate, a phosphor protection film, and the like. In the radiation detection panel 1, some or all of the photoelectric conversion elements (sensors) are an effective imaging region. The effective imaging region is a region where radiation imaging can be performed and an image is actually generated. Although in the present embodiment, the effective imaging region is approximately rectangular when viewed in a radiation incident direction (a side A in FIG. 1), the present disclosure is not limited to this. The phosphor protection film is composed of a material having low moisture permeability and is used to protect the phosphor. The radiation detection panel 1 is electrically connected to one end of a flexible circuit substrate 4a. Further, a control substrate 5 as a first control unit that reads a detection signal from the radiation detection panel 1 and processes the read detection signal is electrically connected to the other end of the flexible circuit substrate 4a. The control substrate 5 includes a control unit and a storage unit (not illustrated). A wireless power reception unit 110 is electrically connected to a charging circuit substrate 7 as a second control unit that controls a charging operation of the wireless power reception unit 110 via a connection cable 4b.

The radiation imaging apparatus 100 includes a housing (exterior) 8 containing the radiation detection panel 1. The radiation detection panel 1 may be of a so-called direct conversion type composed of a conversion element unit in which conversion elements composed of amorphous selenium (a-Se) or the like and electric elements such as thin-film transistors (TFTs) or the like are two-dimensionally arranged, but is not limited to this. As the material of the sensor substrate of the radiation detection panel 1, glass, a highly flexible resin, or the like is possible. The present disclosure, however, is not limited to these. A radiation detector is formed by connecting the flexible circuit substrate 4a, the control substrate 5, a driving substrate (not illustrated), and the like to the radiation detection panel 1. That is, a panel portion of the radiation detector is equivalent to the radiation detection panel 1.

The housing 8 includes a thick portion 8a having an outer shape thick in the radiation incident direction A, and a thin portion 8b having an outer shape thinner than an outer shape of the thick portion 8a. The thick portion 8a and the thin portion 8b are integrated together in the internal space of the housing 8. In the thin portion 8b, the effective imaging region of the radiation detection panel 1 is disposed when viewed in the radiation incident direction A. At least a part of the control substrate 5 is disposed in the thick portion 8a.

Conventionally, the radiation imaging apparatus 100 is often provided in a size compliant with International Organization for Standardization (ISO) 4090:2001 and is often formed with a thickness of about 15 millimeters (mm) to 16 mm. In the present embodiment, however, the thickness of the housing 8 in the thin portion 8b is about 8.0 mm, for example. Thus, when imaging is performed, a difference in level that occurs due to the thickness of the radiation imaging apparatus 100 is small, and therefore, it is possible to ease a reaction force generated in a patient and an end portion of the radiation imaging apparatus 100. To obtain this effect, the thickness of the housing 8 in the thin portion 8b is not limited to about 8.0 mm, and may be thinner. It is confirmed that particularly, when the thickness of the housing 8 in the thin portion 8b is thinner than about 10.0 mm, the effect is obtained.

To achieve both portability and strength, the housing 8 is desirably formed of a magnesium alloy, an aluminum alloy, a fiber-reinforced plastic, a resin, or the like, but may be formed of a material other than these. Particularly, a surface of the thin portion 8b on which the effective imaging region is disposed and radiation is incident is desirably formed of a carbon fiber-reinforced plastic or the like having high radiation transmittance and having excellent lightweight properties, but may be formed of a material other than these. Buffer materials 2 are disposed between the radiation detection panel 1 and an incident surface of the housing 8 and protect the radiation detection panel 1 from an external force or the like. The buffer materials 2 are desirably formed of a foamed resin, a gel, or the like, but may be formed of a material other than these. The radiation imaging apparatus 100 includes a supporting base 3 that supports the radiation detection panel 1. The supporting base 3 is desirably formed of a magnesium alloy, an aluminum alloy, a fiber-reinforced plastic, a resin, or the like having excellent lightweight properties, but may be formed of a material other than these.

On the surface on the incident surface side of the thin portion 8b, an indicator 9 indicating the effective imaging region and a center point of the effective imaging region is provided, and a user can visually identify the effective imaging region using the indicator 9. For example, the indicator 9 is formed by performing surface treatment such as painting, printing, texturing, blasting, or the like on the thin portion 8b, forming a shape such as a depression or the like on the thin portion 8b, or the combination of these.

On the front and back of a central portion of the thick portion 8a, gripping portions 10a and 10b, respectively, having recessed shapes are formed. The user can stably grip the radiation imaging apparatus 100 by putting their pollex on the gripping portion 10a and hooking other fingers on a step portion of the gripping portion 10b. The gripping portion 10a may not be a single continuous recessed surface as illustrated in FIG. 1, and may have a shape such as a rib, a dot, or the like, or may have a protruding shape. Alternatively, a shape may not be provided, and the gripping properties of the gripping portion 10a may be improved by partially using a material having high frictional properties, such as a rubber material or the like, or using surface treatment such as high-friction painting, texturing, or the like. The above shapes, materials, surface treatment, and the like may be appropriately combined together. The gripping portion 10b may also have various shapes, materials, and the like. The arrangement of the gripping portions 10a and 10b may be reversed. A form may be employed in which the gripping portions 10a and 10b are linked together and penetrate the thick portion 8a.

The radiation imaging apparatus 100 includes a battery 6 as a power supply unit that supplies required power. The battery 6 is disposed in the thick portion 8a. As the battery 6, as an example, a lithium-ion battery, an electrical double layer capacitor, an all-solid-state battery, or the like is used, but the battery 6 may be other than these.

The radiation imaging apparatus 100 includes a wireless power transmission/reception unit capable of wirelessly transmitting and receiving power to and from outside. In the present embodiment, a “power transmission/reception unit” refers to a member that performs the supply of power, charging, an imaging operation, the exchange of image information, or the like between the radiation imaging apparatus 100 and outside (an external power supply, a computer, a supporting member, or the like). In view of the fact that a situation mainly described in the present embodiment is a case where, for example, the radiation imaging apparatus 100 is charged from outside, the following describes a case where a wireless power reception unit 110 is provided as the wireless power transmission/reception unit.

The wireless power reception unit 110 includes a wireless coil (a reception side coil) and receives power supplied by a charging apparatus (not illustrated) wirelessly transmitting power. The received power is rectified via the charging circuit substrate 7, then supplied with a predetermined voltage, and used to charge the battery 6 or drive the control substrate 5. The wireless power reception unit 110 is contained in the housing 8 and disposed on a back surface (a surface opposite to a radiation incident surface) side of the radiation detection panel 1 in the thin portion 8b to overlap the radiation detection panel 1 in a plan view. The charging circuit substrate 7 may be disposed in the thick portion 8a or may be disposed in the thin portion 8b. In view of the prevention of unintended reflections due to backscattered radiation (backscattering), however, it is desirable to dispose the charging circuit substrate 7 in the thick portion 8a as illustrated in FIGS. 1 and 2.

The radiation imaging apparatus 100 transmits and receives electromagnetic energy (power) using an electromagnetic induction method as an example of a wireless charging method. Particularly, in the case of the electromagnetic induction method, in a case where the central axis of a power transmission side coil and the central axis of a power reception side coil come as close to each other as possible and ultimately coincide with each other, and the surfaces of the coils come as close to each other as possible, the charging efficiency becomes high. This required accuracy of closeness varies depending on the types of the coils or the surrounding conditions of the coils. The distance between the central axes or the distance between the surfaces is often about 5 mm or less. Although the wireless power reception unit 110 according to the present embodiment includes a single circular coil as illustrated in FIGS. 1 and 2, the present disclosure is not limited to this. The number or the shapes of coils may be changed according to the arrangement in the apparatus or the charging power. For example, a coil having an elliptical or oblong shape may be used. As another wireless charging method, a radio wave reception type or a resonance type may be used.

The supporting base 3 supports the radiation detection panel 1 on an incident surface side of the supporting base 3 and supports the wireless power reception unit 110 on a back surface side of the supporting base 3. The housing 8 further includes a notification unit (not illustrated) composed of a loudspeaker, a light-emitting diode (LED), or the like, and a communication unit (not illustrated) that communicates with a personal computer (PC) or the like using a cable for a wired connection, a wireless local area network (LAN), or the like.

As described above, in the present embodiment, in the radiation imaging apparatus 100 having a small thickness, the wireless power reception unit 110 is disposed on the back surface side of the radiation detection panel 1 in the thin portion 8b. The wireless power reception unit 110 is disposed in the thin portion 8b, thereby preventing an increase in the size of the radiation imaging apparatus 100 due to the provision of the wireless power reception unit 110 so that the radiation imaging apparatus 100 also adapts to a medical cart compatible with a conventional radiation imaging apparatus. The thin portion 8b is disposed on the back surface side of the radiation detection panel 1, whereby the wireless power reception unit 110 is protected by the radiation detection panel 1 and the strength of the wireless power reception unit 110 is improved. This also reduces the possibility of the user touching the wireless power reception unit 110.

### Variations

Variations of the present embodiment are described below.

### First Variation

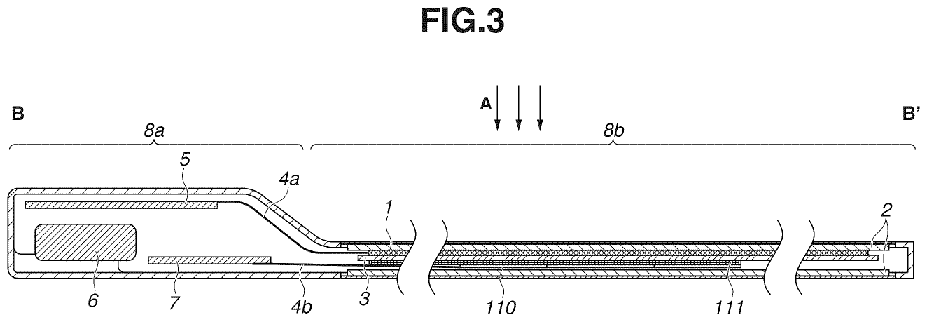

FIG. 3 is a partial cross-sectional view of a radiation imaging apparatus according to a first variation of the present embodiment similar to that in FIG. 2.

The wireless power reception unit 110 and the charging circuit substrate 7 are connected together by a cable or the like. There is a possibility that a defect occurs in control of the radiation detection panel 1 or an artifact is superimposed on a captured image due to the transmission of radiation noise from the wireless power reception unit 110 or the cable to the radiation detection panel 1.

In the first variation, in view of the above issue, a shield material 111 is disposed between the wireless power reception unit 110 or the cable and the radiation detection panel 1. As the shield material 111, a sheet-like material or a film-like material is suitably used. A shield material that exhibits shielding performance against radiation noise having a frequency of several tens of kilohertz to several hundreds of kilohertz, which is suitably used to wirelessly receive and feed power, is suitably used. It is desirable that the shield material 111 be attached to or contained in the supporting base 3. This can reduce the occurrence of erroneous detection (an artifact) due to radiation noise from the wireless power reception unit 110 or the cable.

### Second Variation

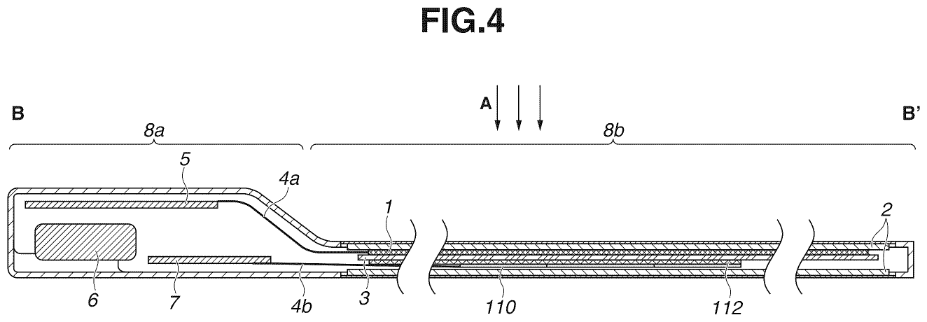

FIG. 4 is a partial cross-sectional view of a radiation imaging apparatus according to a second variation of the present embodiment similar to that in FIG. 2.

The wireless power reception unit 110 or a cable transmits a power of several watts to several tens of watts. Thus, heat is generated due to the impedance of the wireless power reception unit 110 or the cable. Non-uniform temperature distribution may occur in the radiation detection panel 1 due to the transmission of this heat to the radiation detection panel 1. An artifact can be superimposed on a captured image also due to the non-uniform temperature distribution.

In the second variation, in view of the above issue, a thermal insulating material 112 that prevents temperature propagation due to heat generation in the wireless power reception unit 110 or the cable is disposed between the wireless power reception unit 110 or the cable and the radiation detection panel 1. As the thermal insulating material 112, a sheet-like material or a film-like material is suitably used. It is desirable that the thermal insulating material 112 be attached to the supporting base 3 or contained in the supporting base 3. This can reduce the occurrence of an artifact due to heat generation in the wireless power reception unit 110 or the cable.

### Third Variation

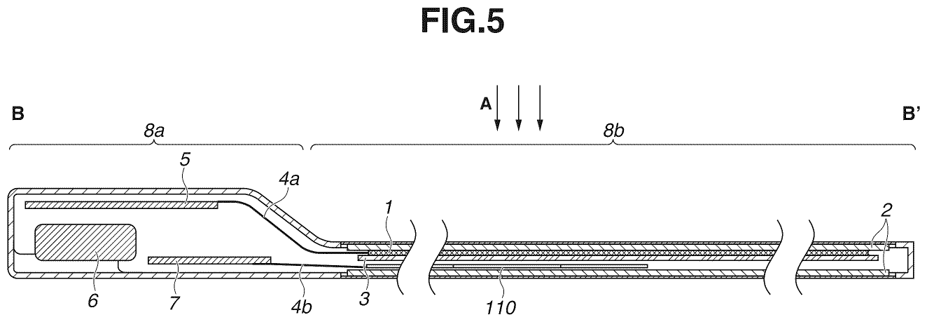

FIG. 5 is a partial cross-sectional view of a radiation imaging apparatus according to a third variation of the present embodiment similar to that in FIG. 2.

In the wireless power reception unit 110 and the charging circuit substrate 7, power loss due to the impedance of the connection cable 4b may occur. Thus, it is desirable to connect the wireless power reception unit 110 and the charging circuit substrate 7 by the connection cable 4b as short a distance as possible. On the other hand, it is desirable to dispose the charging circuit substrate 7 in the thick portion 8a as described above.

In the third variation, in view of the above, the wireless coil of the wireless power reception unit 110 is disposed such that one end of the wireless coil is located in an end portion of the thick portion 8a beyond the outer periphery of the effective imaging region of the radiation detection panel 1 in a plan view. In a case where the wireless coil is disposed at this position, and when the radiation imaging apparatus 100 is accommodated in an accommodation box of a medical cart, one end of the wireless coil is disposed at a place that approximately coincides with an insertion opening of the accommodation box. In the third variation, it is possible to make the distance between the wireless power reception unit 110 and the charging circuit substrate 7 as short as possible and reduce an artifact or power loss.

### Fourth Variation

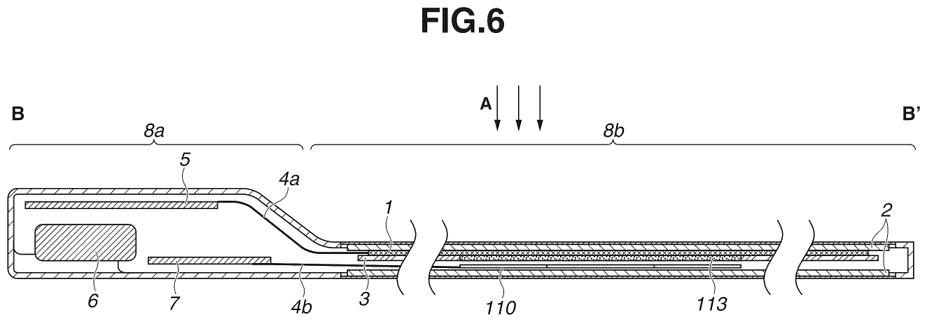

FIG. 6 is a partial cross-sectional view of a radiation imaging apparatus according to a fourth variation of the present embodiment similar to that in FIG. 2.

It is known that the power reception efficiency of the wireless power reception unit 110 changes depending on the material of the supporting base 3. Particularly, if the material of the supporting base 3 includes a metal or another conductive material (an electromagnetic field blocking material), the power efficiency decreases. Thus, it is desirable that the wireless power reception unit 110 be supported by an electromagnetic field transmitting material other than a metal or another conductive material.

In the fourth variation, in view of the above, for example, a portion of the supporting base 3 that supports the wireless power reception unit 110 is molded using an electromagnetic field transmitting material such as a resin material 113. The material only needs to be an electromagnetic field transmitting material, and for example, may be a formed material. A part of the supporting base 3 may be lightened. This can reduce a decrease in the power efficiency.

### Second Embodiment

With reference to the drawings, a second embodiment is described in detail below. Descriptions similar to the first embodiment are appropriately omitted. Although in the first embodiment, an example has been described where the wireless power transmission/reception unit (specifically, the wireless power reception unit 110) is provided in the thin portion 8b, a power transmission/reception unit using a wired line (a wired power transmission/reception unit other than the wireless power transmission/reception unit), in this case, a wired power reception unit, can be provided in addition to the wireless power reception unit 110. The present embodiment is different from the first embodiment in that the wired power reception unit is further included in addition to the wireless power reception unit 110.

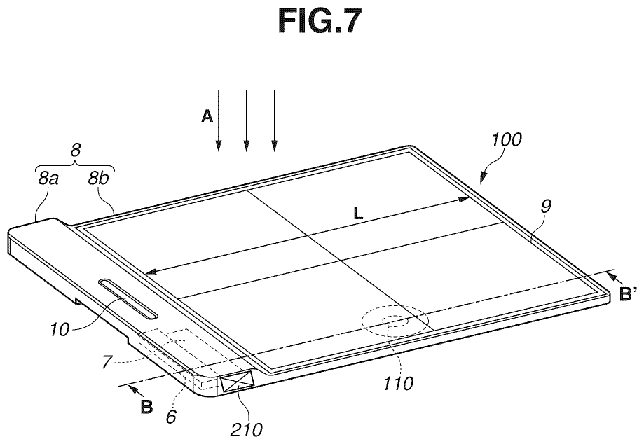

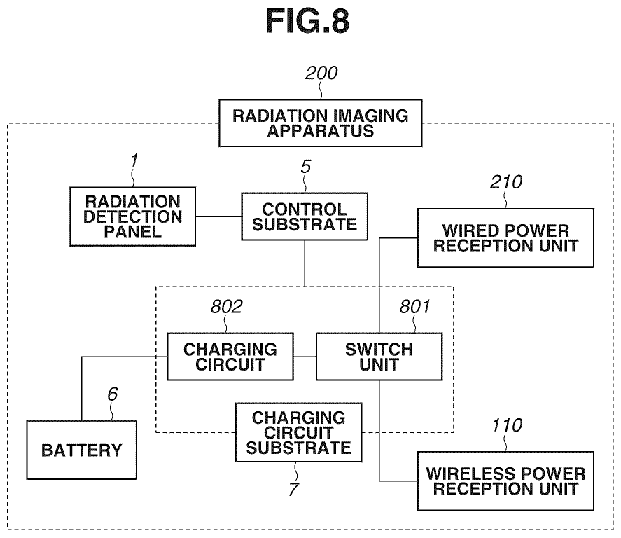

FIG. 7 is a perspective view illustrating a radiation imaging apparatus according to the present embodiment. FIG. 8 is a block diagram illustrating the configuration of the radiation imaging apparatus according to the present embodiment.

In a radiation imaging apparatus 200 according to the present embodiment, a wired power reception unit 210 is disposed on one side surface of the thick portion 8a in addition to the wireless power reception unit 110 disposed in the thin portion 8b similarly to the first embodiment. As the wired power reception unit 210, a power transmission/reception connector, in this case, a general-purpose or dedicated connector or the like, is suitably used. The wired power reception unit 210 receives power via a wired connection from a charging apparatus (not illustrated).

The wireless power reception unit 110 and the wired power reception unit 210 are connected to a charging circuit 802 via a switch unit 801 in the charging circuit substrate 7. Power received by the charging circuit 802 is rectified, then supplied with a predetermined voltage, and used to charge the battery 6 or drive the control substrate 5. The control substrate 5 monitors the power transmission/reception states, in this case, the power reception states, of the wireless power reception unit 110 and the wired power reception unit 210 with the switch unit 801 and switches a power reception unit connected to the charging circuit 802 between the wireless power reception unit 110 and the wired power reception unit 210 from one to the other under a predetermined condition. It is desirable to determine the predetermined condition in advance and store the predetermined condition in the control substrate 5. Although the predetermined condition may be any condition, for example, a condition for switching the power reception unit based on the connection state by setting the following priorities is suitably used. That is, in a case where the wireless power reception unit 110 can feed power, the wireless power reception unit 110 is connected, and when the feeding of power from the wireless power reception unit 110 is disrupted, the power reception unit is switched to the wired power reception unit 210.

### Third Embodiment

With reference to the drawings, a third embodiment is described in detail below. Descriptions similar to the first embodiment are appropriately omitted. Although in the first embodiment, an example has been described where the wireless power transmission/reception unit (specifically, the wireless power reception unit 110) is provided in the thin portion 8b, a power transmission/reception unit using a wired line (a wired power transmission/reception unit), in this case, a wired power reception unit, can be provided instead of the wireless power reception unit 110. The present embodiment is different from the first embodiment in that the wired power reception unit is further included instead of the wireless power reception unit 110.

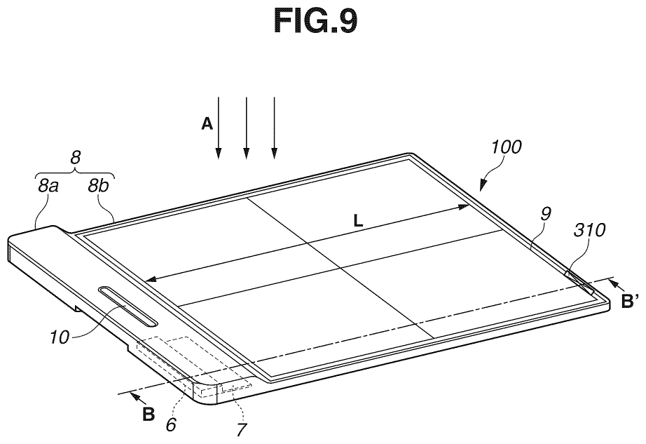

FIG. 9 is a perspective view illustrating a radiation imaging apparatus according to the present embodiment.

In a radiation imaging apparatus 300 according to the present embodiment, a wired power reception unit 310 is disposed on a side surface of the thin portion 8b facing the longitudinal direction of the thin portion 8b, instead of the wireless power reception unit 110 of the radiation imaging apparatus 100 according to the first embodiment. The wired power reception unit 310 receives power via a wired connection from a charging apparatus (not illustrated).

As the wired power reception unit 310, a connector having a small thickness and capable of transmitting a power of several watts to several tens of watts is suitably used in view of the influence of the wired power reception unit 310 on the thickness of the thin portion 8b. For example, a Universal Serial Bus (USB) connector or the like is suitably used. Particularly, a USB Type-C connector having a thickness of about several millimeters is suitably used. In consideration of the handleability of a wired power transmission apparatus and a cable, it is desirable to dispose the wired power reception unit 310 at a position that does not overlap the radiation detection panel 1 in a plan view, such as a side surface of the thin portion 8b. The wired power reception unit 310 may be disposed on the back surface (the surface opposite to the radiation incident surface) side of the radiation detection panel 1 in the thin portion 8b to overlap the radiation detection panel 1 in a plan view.

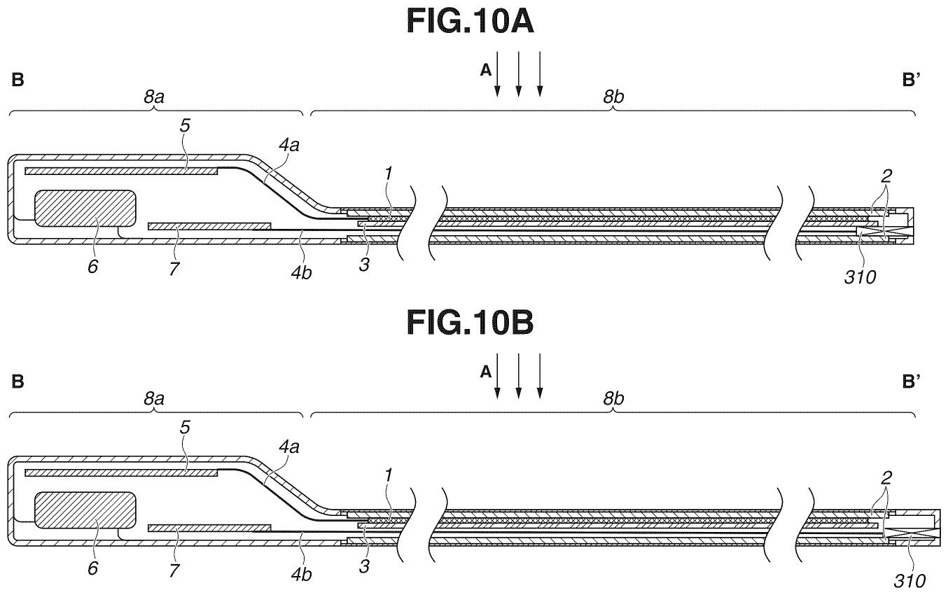

FIGS. 10A and 10B are partial cross-sectional views of the radiation imaging apparatus according to the present embodiment similar to that in FIG. 2.

In FIGS. 10A and 10B, the wired power reception unit 310 is a so-called USB Type-C connector. The wired power reception unit 310 is disposed such that an opening portion of the wired power reception unit 310 is located in the thin portion 8b. In FIG. 10A, at least a part of a housing of the wired power reception unit 310 is disposed on the back surface side of the radiation detection panel 1. Consequently, it is possible to expect the prevention of an increase in the size of the outer shape of the radiation imaging apparatus 300 and an improvement in the strength of the housing 8 due to the provision of the wired power reception unit 310. In FIG. 10B, the housing of the wired power reception unit 310 is disposed outside the radiation detection panel 1. This can prevent an increase in the thickness of the thin portion 8b due to the provision of the wired power reception unit 310.

### Fourth Embodiment

With reference to the drawings, a fourth embodiment is described in detail below. Descriptions similar to the first embodiment are appropriately omitted. Although in the second variation of the first embodiment, an example has been described where the influence of temperature is addressed by installing a thermal insulating material as countermeasures, other measures are also possible. The fourth embodiment is different from the second variation of the first embodiment in that charging is controlled using a temperature sensor.

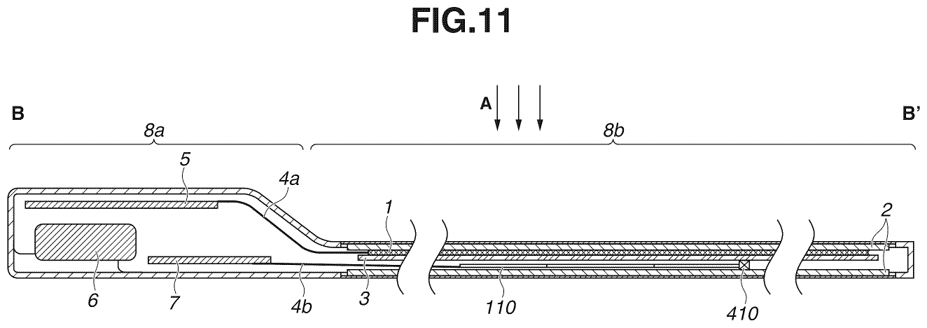

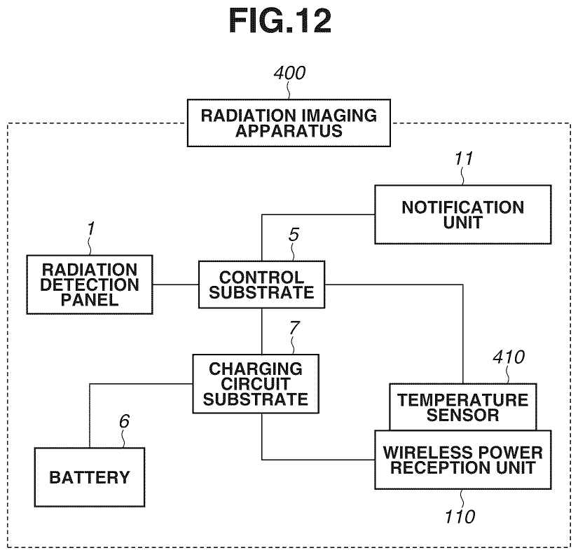

FIG. 11 is a partial cross-sectional view of a radiation imaging apparatus according to the present embodiment similar to that in FIG. 2. FIG. 12 is a block diagram illustrating the configuration of the radiation imaging apparatus according to the present embodiment.

In a radiation imaging apparatus 400 according to the present embodiment, a temperature sensor 410 is disposed in the proximity of the wireless power reception unit 110. The control substrate 5 monitors changes in temperature according to charging measured by the temperature sensor 410 and performs control according to temperature. A notification unit 11 notifies the outside of information generated by the control substrate 5 monitoring temperature.

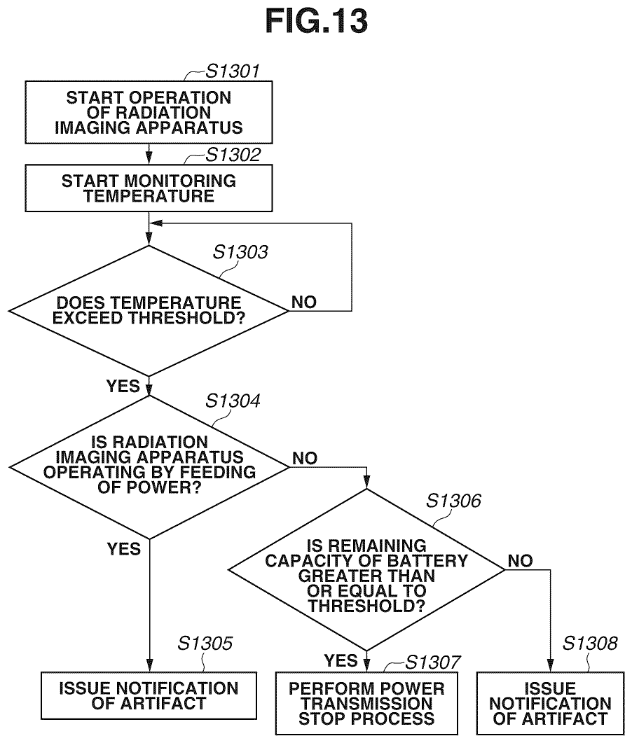

FIG. 13 is a flowchart illustrating an example of the control according to temperature according to the present embodiment.

In step S1301, the operation of the radiation imaging apparatus 300 is started. In step S1302, the control substrate 5 starts monitoring temperature using the temperature sensor 410.

In step S1303, the control substrate 5 determines whether temperature detected by the temperature sensor 410 exceeds a threshold. It is desirable to determine the threshold in advance and store the threshold in a storage unit (not illustrated) or the like mounted on the control substrate 5. In a case where the detected temperature exceeds the threshold (YES in step S1303), the processing proceeds to step S1304. In a case where the detected temperature does not exceed the threshold (NO in step S1303), the processing returns to step S1302. In step S1302, the control substrate 5 continues to monitor and determine temperature.

In step S1304, the control substrate 5 determines whether the radiation imaging apparatus 300 is operating by the feeding of power from the wireless power reception unit 110. In a case where the feeding of power from the wireless power reception unit 110 is being used for the operation of the radiation imaging apparatus 400 (YES in step S1304), the processing proceeds to step S1305. In step S1305, the notification unit 11 issues a notification of cautionary information regarding an artifact or the like due to temperature. In a case where the feeding of power from the wireless power reception unit 110 is not being used for the operation of the radiation imaging apparatus 400 (NO in step S1304), the processing proceeds to step S1306.

In step S1306, the control substrate 5 determines whether the remaining capacity of the battery 6 is greater than or equal to a threshold. It is desirable to determine the threshold in advance and store the threshold in a storage unit (not illustrated) or the like mounted on the control substrate 5. As the threshold, a capacity capable of maintaining a power supply for a certain time, a capacity that enables a certain number of images to be captured, or the like is suitably used. In a case where the remaining capacity of the battery 6 is greater than or equal to the threshold (YES in step S1306), the processing proceeds to step S1307.

In step S1307, the control substrate 5 notifies a wireless power transmission unit of power transmission stop information via the wireless power reception unit 110. Consequently, the control substrate 5 stops the transmission and reception of power to and from the wireless power transmission/reception unit and prevents a rise in temperature. In a case where the remaining capacity of the battery 6 is not greater than or equal to the threshold (NO in step S1306), the processing proceeds to step S1308. In step S1308, the notification unit 11 issues a notification of cautionary information regarding an artifact or the like due to temperature.

As described above, in the present embodiment, the temperature sensor 410 is provided and monitors temperature, whereby it is possible to reduce the occurrence of an artifact due to heat generation in the wireless power reception unit 110 or the cable.

### Fifth Embodiment

With reference to the drawings, a fifth embodiment is described in detail below. Descriptions similar to the first embodiment are appropriately omitted. Although in the fourth embodiment, an example of a method for addressing the influence of temperature using a temperature sensor has been described, other measures are also possible. The fifth embodiment is different from the fourth embodiment in that the influence of temperature is addressed using time information.

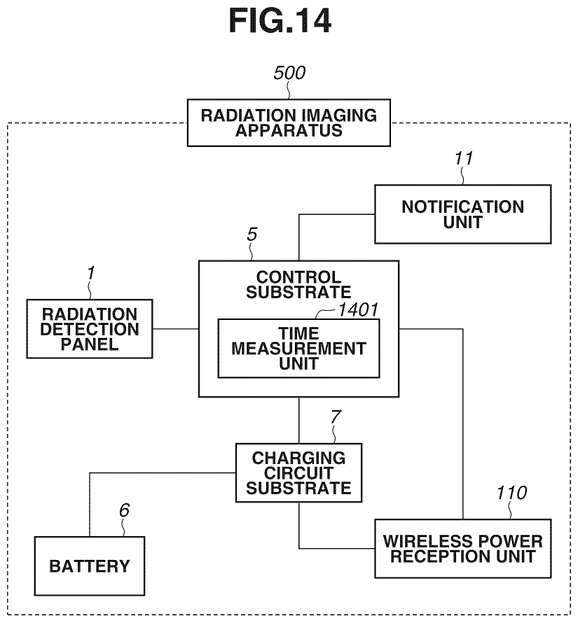

FIG. 14 is a block diagram illustrating the configuration of a radiation imaging apparatus according to the present embodiment.

In a radiation imaging apparatus 500 according to the present embodiment, a time measurement unit 1401 that measures the time elapsed during the reception of power (the time of the feeding of power from the wireless power reception unit 110) or the time elapsed from the end of the reception of power (the completion of the charging of the battery 6) is provided in the control substrate 5. The notification unit 11 notifies the outside of information generated by the control substrate 5 measuring time.

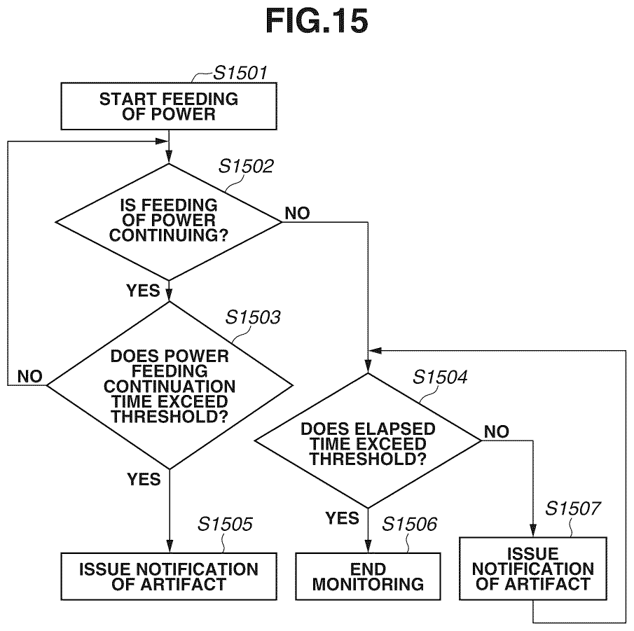

FIG. 15 is a flowchart illustrating an example of the control according to time according to the present embodiment.

In step S1501, when the feeding of power from the wireless power reception unit 110 is started, the control substrate 5 starts measuring time using the time measurement unit 1401. In step S1502, the control substrate 5 determines a power feeding state regarding whether a wireless power transmission unit that transmits power to the wireless power reception unit 110 is detached, or whether the charging of the battery 6 is completed and the feeding of power stops. In a case where the feeding of power is continuing (YES in step S1502), the processing proceeds to step S1503. In a case where the feeding of power stops (NO in step S1502), the processing proceeds to step S1504.

In step S1503, the control substrate 5 determines whether a power feeding continuation time exceeds a threshold. It is desirable to determine the threshold in advance by determining the influence on a rise in temperature, an artifact, or the like according to the feeding of power or the charging of the battery 6. It is desirable to store the threshold in a storage unit (not illustrated) or the like mounted on the control substrate 5. In a case where the power feeding continuation time exceeds the threshold (YES in step S1503), the processing proceeds to step S1505. In step S1505, the notification unit 11 issues a notification of cautionary information regarding an artifact or the like due to temperature. In a case where the power feeding continuation time does not exceed the threshold (NO in step S1503), the processing returns to step S1502. In step S1502, the control substrate 5 continues the monitoring.

In step S1504, the control substrate 5 determines whether the time elapsed from the end of the feeding of power exceeds a threshold. It is desirable to determine the threshold in advance by determining the amount of rise in temperature during the feeding of power, the amount of fall in temperature from the end of the feeding of power, or the like. It is desirable to store the threshold in a storage unit (not illustrated) or the like mounted on the control substrate 5.

In a case where the time elapsed from the end of the feeding of power exceeds the threshold (YES in step S1504), then in step S1506, the control substrate 5 determines that there is NO concern for an artifact or the like. Then, the control substrate 5 ends the monitoring. In a case where the time elapsed from the end of the feeding of power does not exceed the threshold (is less than the threshold) (NO in step S1504), the processing proceeds to step S1507. In step S1507, the notification unit 11 issues a notification of cautionary information regarding an artifact or the like due to temperature. Then, the processing returns to step S1504. In step S1504, the control substrate 5 continues the determination until the time elapsed from the end of the feeding of power exceeds the threshold.

As described above, in the present embodiment, the time measurement unit 1401 is provided and monitors temperature based on measured time, whereby it is possible to reduce the occurrence of an artifact due to heat generation in the wireless power reception unit 110 or the cable.

### Other Embodiments

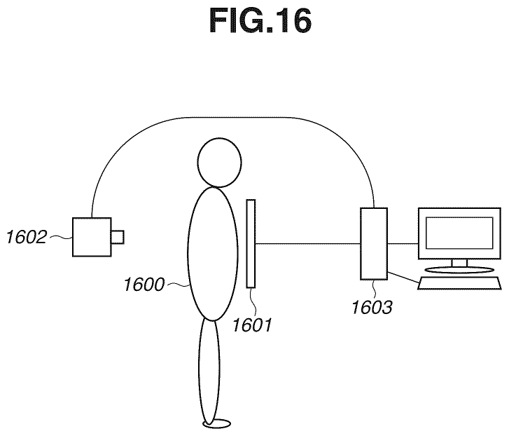

For example, the radiation imaging apparatuses according to the first to fifth embodiments and the variations can be applied to a radiation imaging system as illustrated in FIG. 16.

This radiation imaging system includes a radiation imaging apparatus 1601 as one of the radiation imaging apparatuses according to the first to fifth embodiments and the variations, a radiation generating apparatus 1602, and a control/calculation processing unit 1603. The radiation imaging apparatus 1601 and the radiation generating apparatus 1602 are connected to the control/calculation processing unit 1603. Under control of the control/calculation processing unit 1603, the radiation generating apparatus 1602 emits radiation to a subject 1600. The radiation imaging apparatus 1601 detects the radiation transmitted through the subject 1600. Information detected by the radiation imaging apparatus 1601 is read as an electric signal by the control/calculation processing unit 1603. The control/calculation processing unit 1603 performs a desired calculation process and performs a diagnosis.

### Sixth Embodiment

In a sixth embodiment, the following describes examples of a radiation imaging apparatus, an accommodation member for the radiation imaging apparatus, and a power reception/feeding system of the radiation imaging apparatus to which the present disclosure is applied.

### [Radiation Imaging Apparatus According to Present Embodiment]

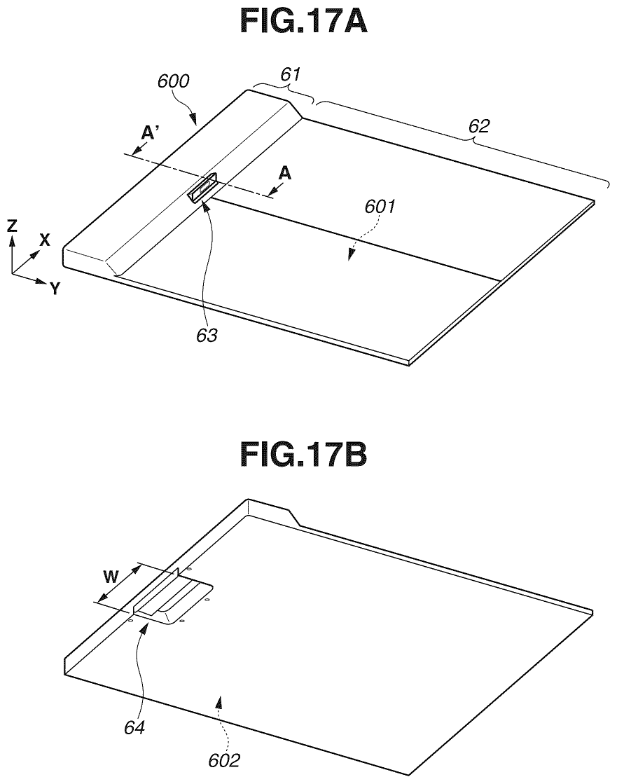

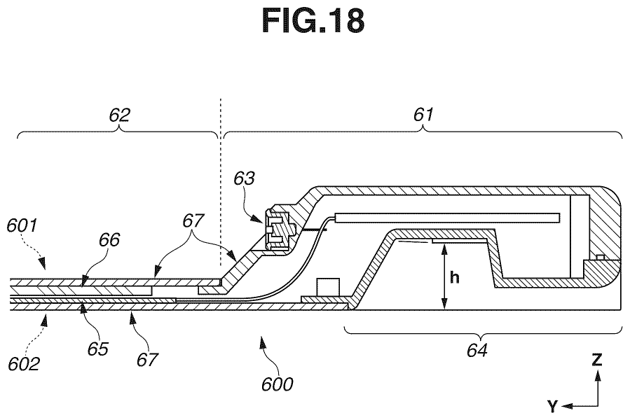

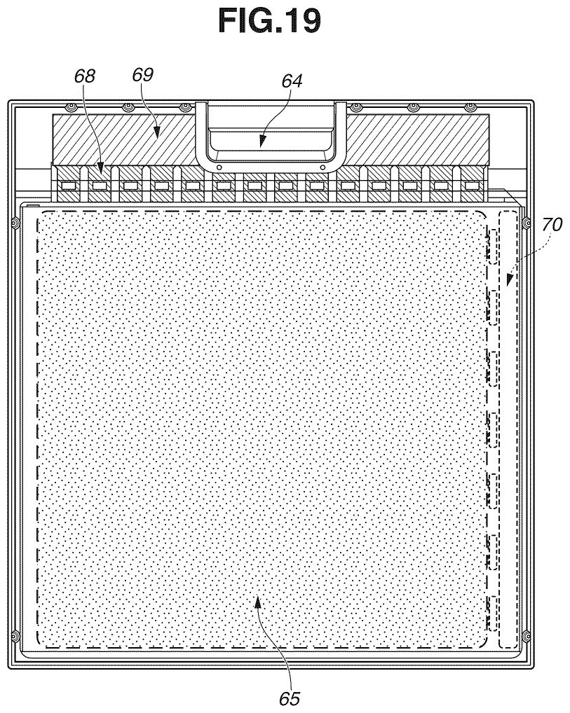

FIGS. 17A and 17B are perspective views illustrating a radiation imaging apparatus according to the present embodiment. FIG. 17A illustrates a front surface side, and FIG. 17B illustrates a back surface side. FIG. 18 is a cross-sectional view along a dashed-dotted line A-A′ in FIG. 17A. FIG. 19 is a plan view of the back surface side illustrating the internal configuration of the radiation imaging apparatus according to the present embodiment. In the following description, in the radiation imaging apparatus, a surface on the side where the radiation imaging apparatus is irradiated with radiation (an irradiation surface) is a front surface 601, and a surface opposite to the irradiation surface is a back surface 602.

A radiation imaging apparatus 600 detects radiation emitted from a radiation generating apparatus (not illustrated) and transmitted through an object, using a radiation detection panel 65. An image acquired by the radiation imaging apparatus 600 is transferred to the outside, displayed on a monitor or the like, and used for a diagnosis or the like.

The radiation detection panel 65 includes a sensor substrate on which many photoelectric conversion elements (photoelectric conversion sensors) that convert incident radiation into electric signals regarding a radiation image are disposed, a phosphor layer (a scintillator layer) disposed on the sensor substrate, a phosphor protection film, and the like. The radiation detection panel 65 employs a so-called indirect conversion method. In the radiation detection panel 65, a region of some or all of the photoelectric conversion elements is an effective imaging region. The effective imaging region is a region where radiation imaging can be performed and an image is actually generated. The phosphor protection film is composed of a material having low moisture permeability and is used to protect the phosphor. The radiation detection panel 65 is connected to a flexible circuit substrate 68. Further, a control substrate 69 that reads a detection signal from the radiation detection panel 65 and processes the read detection signal is connected to the flexible circuit substrate 68. A driving substrate 70 including a driving circuit on a side intersecting the control substrate 69 is also connected to the radiation detection panel 65. A radiation detector is formed by connecting the circuit substrate 68, the control substrate 69, and the driving substrate 70 to the radiation detection panel 65. A panel portion of the radiation detector is equivalent to the radiation detection panel 65.

In a conventional radiation imaging apparatus, a control substrate and a driving substrate are installed in a folded manner on a back surface side of a radiation detection panel. In contrast, in the radiation imaging apparatus 600 according to the present embodiment, to achieve an extremely thin shape, the control substrate 69 and the driving substrate 70 are provided side by side on the radiation detection panel 65 without being folded on the back surface side of the radiation detection panel 65. The driving substrate 70 is disposed on approximately the same plane as the radiation detection panel 65. The control substrate 69 can also be disposed on approximately the same plane as the radiation detection panel 65, but interferes with a gripping portion 64 disposed on the back surface 602. Thus, the control substrate 69 is disposed in a thick portion 61 to avoid the gripping portion 64.

As the material of the sensor substrate of the radiation detection panel 65, a variety of materials such as glass, a highly flexible resin, and the like can be used. It is desirable to use a base material formed of a resin film. The radiation imaging apparatus 600 according to the present embodiment is thinner than the conventional radiation imaging apparatus. Thus, basically, the radiation imaging apparatus 600 has a low strength and is likely to be influenced by an external shock or the like. Thus, a resin film, which exhibits superior resistance to impact and bending compared to a glass substrate, is suitable for use.

The radiation imaging apparatus 600 includes a housing 67 containing the radiation detector including the radiation detection panel 65, the circuit substrate 68, the control substrate 69, and the driving substrate 70. In the internal space of the housing 67, a supporting base (not illustrated) that supports the radiation detection panel 65, a shock absorption sheet 66 that softens a shock, and the like are disposed. To achieve both portability and strength, the material of the housing 67 is desirably formed of a magnesium alloy, a fiber-reinforced plastic, a resin, or the like, but is not limited to these. The material of the shock absorption sheet 66 is desirably formed of a foamed resin, a gel, or the like, but is not limited to these. Although in the present embodiment, the supporting base is used, the radiation detection panel 65 may be disposed without using the supporting base. The housing 67 may be formed by combining a plurality of components, or may be formed of a single component.

In the radiation imaging apparatus 600, as illustrated in FIGS. 17A and 17B, the housing 67 includes a thick portion 61 and a thin portion 62 integrated together in the internal space. As illustrated in FIG. 18, the thickness of the thick portion 61 is a part thicker than the thickness of the thin portion 62 in the state where the thick portion 61 is installed on an X-Y plane. The thick portion 61 also includes a sloping portion from an end portion of the thin portion 62 to the thickest portion. The radiation detector is contained in the internal space of the housing 67. The portion of the circuit substrate 68 and the control substrate 69 are disposed in the internal space of the thick portion 61, and the panel portion (the portion of the radiation detection panel 65) is disposed in the internal space of the thin portion 62.

In the present embodiment, in the radiation detector accommodated in the housing 67, the circuit substrate 68 and the control substrate 69 in the thick portion 61 are connected (joined) to the radiation detection panel 65 in the thin portion 62. With this configuration, even if the radiation imaging apparatus 600 receives a shock when the radiation imaging apparatus 600 is accommodated in a predetermined accommodation member, breakage or the like is less likely to occur in this joint portion in comparison with a case where the radiation detection panel 65, the control substrate 69, and the like are configured as separate components.

Normally, a radiation imaging apparatus is often provided in a size compliant with ISO 4090:2001, and the thickness of the radiation imaging apparatus is defined as about 15 mm to about 16 mm. In contrast, in the present embodiment, the thickness of the thin portion 62 is about 8.0 mm. Thus, thickness due to the difference in level between the radiation imaging apparatus 600 and a contact surface is thin, and therefore, it is possible to ease a reaction force generated in a patient and an end portion of the radiation imaging apparatus 600. To obtain this effect, the thickness of the thin portion 62 is not limited to about 8.0 mm, and may be thinner. It is confirmed that particularly, in a case where the thickness of the thin portion 62 is thinner than about 10.0 mm, a significant effect is obtained. In the present embodiment, thinning is achieved by aggregating internal components having a certain level of thickness, such as substrates, a battery, and the like (not illustrated), in the thick portion 61 wherever possible.

In the radiation imaging apparatus 600, a recessed gripping portion 64 for, for example, carrying around the radiation imaging apparatus 600 is provided in the thick portion 61. For example, the gripping portion 64 is formed by providing a recessed portion in a portion on the back surface 602 side of the thick portion 61 of the housing 67. The gripping portion 64 is provided in the thick portion 61, whereby it is easy for the user to handle the radiation imaging apparatus 600 when the user performs the work of inserting and removing the radiation imaging apparatus 600 immediately below a supine subject, or the like. Thus, the user can quickly perform radiation imaging. In terms of the above workability, it is desirable that the gripping portion 64 be formed on the back surface 602. Although the shape of the gripping portion 64 is not limited to the illustrated shape, it is desirable that, for example, a width W of the gripping portion 64 should be about 60 mm or more, and a depth h of the gripping portion 64 should be about 5 mm or more, assuming that the user hooks their fingers on the gripping portion 64.

In the radiation imaging apparatus 600, a power reception/feeding unit capable of receiving and feeding power from and to outside is provided in the thick portion 61. For example, a power reception connector 63 is disposed as a power reception unit capable of feeding power to the radiation detector from outside. The power reception connector 63 can supply power to or charge the radiation imaging apparatus 600 by being connected to an external power feeding connector. In the present embodiment, a “power reception/feeding unit” refers to a member that performs the supply of power, charging, an imaging operation, the exchange of image information, or the like between the radiation imaging apparatus 600 and outside (an external power supply, a computer, a supporting member, or the like). In view of the fact that a situation mainly described in the present embodiment is a case where, for example, the radiation imaging apparatus 600 is charged from outside, a power reception/feeding unit provided in the radiation imaging apparatus 600 is referred to as a “power reception unit”, and a power reception/feeding unit provided outside is referred to as a “power feeding unit” for convenience.

The power reception connector 63 is disposed and embedded in the layer of the housing 67 in the thick portion 61, and a connection portion of the power reception connector 63 with the power feeding connector is exposed to the outside through the surface of the housing 67. In the present embodiment, the power reception connector 63 is disposed in the sloping portion as a boundary part of the thick portion 61 with the thin portion 62. Although in the examples of FIGS. 17A and 17B, the power reception connector 63 is disposed in an approximately central part of the sloping portion of the thick portion 61, for example, in view of usability, the power reception connector 63 may be disposed at a position shifted from the central part of the sloping portion to avoid overlap with the gripping portion 64 on the back surface 602 in a plan view.

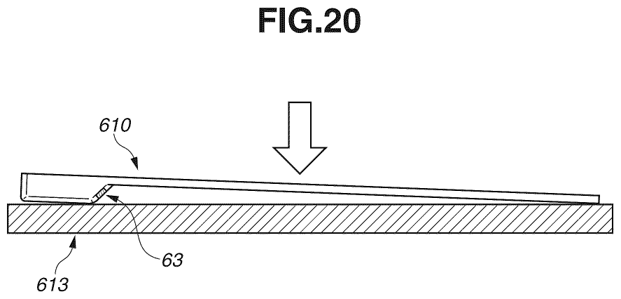

In the conventional radiation imaging apparatus compliant with ISO 4090, a power reception connector is provided on a side surface having a thickness of about 15 mm to about 16 mm. However, in the radiation imaging apparatus 600 according to the present embodiment, since the thickness of the thin portion 62 is thin, namely about 8.0 mm, it is difficult to dispose a power reception connector on a side surface of the thin portion 62. Even if the power reception connector is forcedly disposed on the side surface, there is a high possibility that the power reception connector disposed in the thin portion 62 is broken. Particularly, since the thin portion 62 has a low strength, a connection portion between the power reception connector and the power feeding connector cannot bear bending stress and may be broken. Accordingly, in the present embodiment, the power reception connector 63 is disposed in the thick portion 61. The thick portion 61 has a high stiffness compared to the thin portion 62 and can reduce the risk of the breakage of the power reception connector 63. Specifically, the power reception connector 63 is disposed in the sloping portion of the thick portion 61. The power reception connector 63 is disposed in the sloping portion, whereby, as illustrated in FIG. 20, even if the radiation imaging apparatus 600 is inadvertently dropped on a floor 613, the power reception connector 63 is in a non-contact state with the floor 613. Thus, the breakage of the power reception connector 63 is avoided.

According to the present embodiment, a radiation imaging apparatus is achieved in which, even if the radiation imaging apparatus receives a shock when the radiation imaging apparatus is carried around or the like, breakage is less likely to occur, and it is possible to easily and certainly receive and feed power with high reliability by electrically connecting the power reception connector 63 disposed in the thick portion 61 of the housing 67 to the external power feeding connector.

### [Supporting Member According to Present Embodiment]

For example, the radiation imaging apparatus according to the present embodiment can be carried by being accommodated in an accommodation box of a medical cart. At this time, it is possible to feed power to the radiation imaging apparatus while the radiation imaging apparatus is mounted on the medical cart. It is also possible to put the radiation imaging apparatus into not only a medical cart but also a power feeding stand dedicated to power feeding and feed power to the radiation imaging apparatus. Further, it is also possible that the radiation imaging apparatus is put into a bucky stand or the like, and radiation imaging is performed while power is fed to the radiation imaging apparatus. Although an operation of putting the radiation imaging apparatus into and taking the radiation imaging apparatus out of an accommodation unit is performed in any case, the radiation imaging apparatus according to the present embodiment includes a thin portion and a thick portion, and the position of a power reception connector is also greatly different from that in the conventional radiation imaging apparatus. Thus, the configuration of an accommodation member also needs to be greatly changed from a conventional configuration.

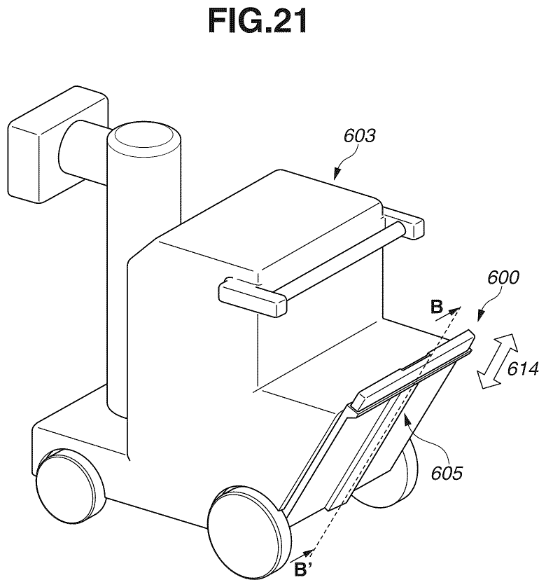

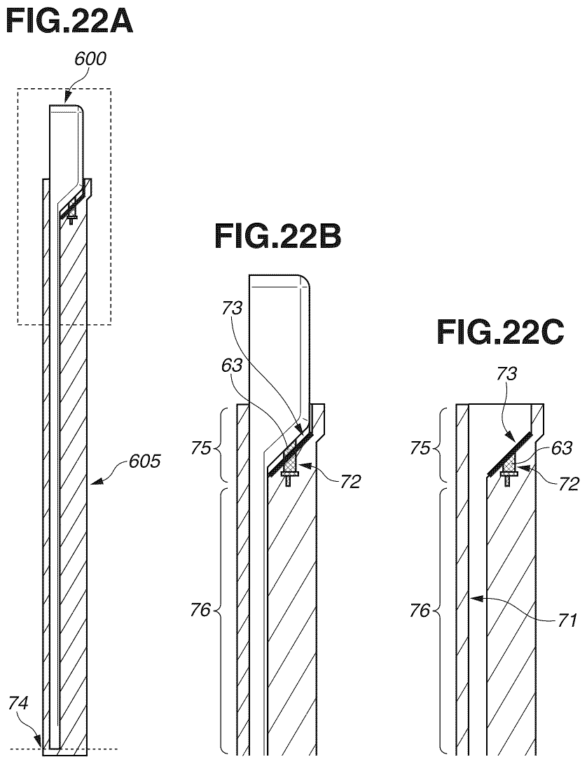

The following describes an accommodation box of a medical cart as an example of an accommodation member that accommodates the radiation imaging apparatus according to the present disclosure. FIG. 21 is a perspective view illustrating a medical cart including an accommodation box for the radiation imaging apparatus according to the present embodiment. FIGS. 22A to 22C are cross-sectional views of the accommodation box along a dashed-dotted line B-B′ in FIG. 21. FIG. 22A illustrates the state where the radiation imaging apparatus is accommodated in the accommodation box. FIG. 22B illustrates an enlarged view of the inside of a dashed rectangular frame in FIG. 22A. FIG. 22C illustrates the state where the radiation imaging apparatus is not accommodated in an accommodation unit in FIG. 22B. The internal structure of an accommodation box or the like as an accommodation member is referred to as an “accommodation unit”.

In the radiation imaging apparatus 600 accommodated in an accommodation box 605, in the radiation detector accommodated in the housing 67, the circuit substrate 68 and the control substrate 69 in the thick portion 61 are connected (joined) to the radiation detection panel 65 in the thin portion 62. Thus, even if the radiation imaging apparatus 600 receives a shock when the radiation imaging apparatus 600 is accommodated in the accommodation box 605, breakage or the like is less likely to occur in the above joint portion in comparison with in a radiation imaging apparatus in which the radiation detection panel 65, the control substrate 69, and the like are configured as separate components.

In a case where the radiation imaging apparatus 600 is put into and taken out of the accommodation box 605 of a medical cart 603, the user performs this operation by holding the gripping portion 64 provided in the thick portion 61. Thus, the radiation imaging apparatus 600 is inserted into an accommodation unit 71 with the thin portion 62 first. If the size of the accommodation unit 71 is excessively large, the thin portion 62 collides with a bottom surface 74 of the accommodation unit 71, and the thin portion 62 of the radiation imaging apparatus 600 may be damaged by a drop impact or the like. As described above, even in the radiation imaging apparatus 600 including the radiation detector according to the present embodiment taking into account the ensuring of the strength of the joint portion, the thin portion 62 has a weak strength and may be broken by a shock. Thus, it is desirable that the accommodation unit 71 of the radiation imaging apparatus 600 be configured to contacts the thick portion 61. Accordingly, in the present embodiment, in the accommodation unit 71 that accommodates the radiation imaging apparatus 600, a supporting surface 73 is provided as a supporting portion capable of supporting at least the thick portion 61.

The accommodation unit 71 includes a thick region 75 and a thin region 76. The thick region 75 corresponds to the accommodation of the thick portion 61 of the radiation imaging apparatus 600, and the thin region 76 corresponds to the accommodation of the thin portion 62 of the radiation imaging apparatus 600. In the accommodation box 605, the sloping portion of the thick portion 61 of the radiation imaging apparatus 600 and the supporting surface 73 of the thick region 75 come into contact with each other. A configuration is employed in which, when the radiation imaging apparatus 600 is accommodated in the accommodation box 605, the sloping portion of the thick portion 61 is received by the supporting surface 73 as a sloping surface corresponding to the sloping portion, whereby a shock is softened. The accommodation box 605 is not limited to a configuration in which the accommodation box 605 supports the sloping portion of the thick portion 61, and a supporting surface may be provided in the accommodation box 605 to support a side surface of the thick portion 61. Further, for example, the accommodation box 605 may be configured to support the gripping portion 64 on the back surface side of the radiation imaging apparatus 600.

In the present embodiment, the power reception connector 63 is provided in the sloping portion of the thick portion 61 of the radiation imaging apparatus 600. On the supporting surface 73 of the accommodation unit 71 of the medical cart 603, a power feeding connector 72 is provided as a power reception/feeding unit such as a power feeding unit capable of performing charging or the like when the radiation imaging apparatus 600 is accommodated. In a case where the radiation imaging apparatus 600 is put into the accommodation unit 71 of the medical cart 603, the sloping portion of the thick portion 61 of the radiation imaging apparatus 600 abuts the supporting surface 73 of the accommodation unit 71, and the radiation imaging apparatus 600 is supported in the accommodation unit 71.

In the abutment, the power reception connector 63 disposed in the sloping portion of the thick portion 61 and the power feeding connector 72 disposed in the supporting surface 73 of the accommodation unit 71 are electrically connected together, and power is received and fed between the accommodation box 605 and the radiation imaging apparatus 600.

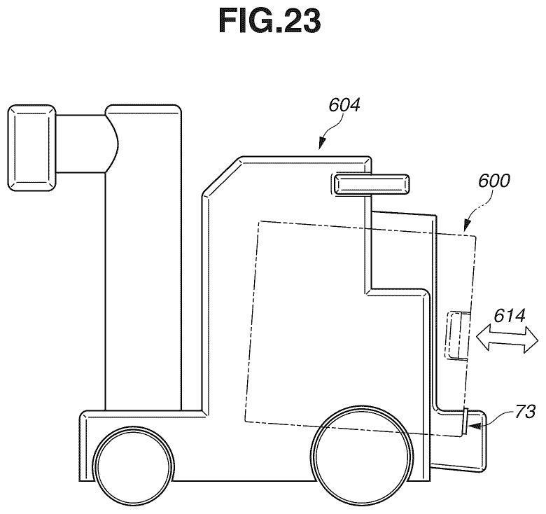

FIG. 23 is a side view illustrating another example of the medical cart including the accommodation box for the radiation imaging apparatus according to the present embodiment.

In the accommodation box 605 of a medical cart 604, the supporting surface 73 is disposed in such a manner that when the radiation imaging apparatus 600 is put into the accommodation unit 71, the supporting surface 73 supports a side surface which is the opposite side of the thin portion 62 side among four side surfaces of the thick portion 61 of the radiation imaging apparatus 600. The accommodation unit 71 slopes downward on the supporting surface 73 side, and therefore, the radiation imaging apparatus 600 is configured to naturally support the supporting surface 73. To increase the strengths of the supporting surface 73 and the housing 67 of the radiation imaging apparatus 600 corresponding to the supporting surface 73, the materials of the supporting surface 73 and the housing 67 are each formed of a metal, or the supporting surface 73 and the housing 67 are partially thickened, or a predetermined rib is provided, whereby the supporting surface 73 and the housing 67 are more resistant to shocks.

In the medical cart 604 illustrated in FIG. 23, the power reception connector 63 is provided on the side surface which is on the opposite side of the thin portion 62 side among the four side surfaces of the thick portion 61 of the radiation imaging apparatus 600. On the supporting surface 73 of the accommodation unit 71 of the medical cart 604, the power feeding connector 72 is provided so that, for example, power can be fed to the radiation imaging apparatus 600 when the radiation imaging apparatus 600 is attached. When the radiation imaging apparatus 600 is put into the accommodation unit 71 of the medical cart 603, the side surface of the thick portion 61 of the radiation imaging apparatus 600 abuts the supporting surface 73 of the accommodation unit 71, and the radiation imaging apparatus 600 is supported in the accommodation unit 71. In the abutment, the power reception connector 63 disposed on the side surface of the thick portion 61 and the power feeding connector 72 disposed in the supporting surface 73 of the accommodation unit 71 are electrically connected together, and power is received and fed. As the reception and feeding of power, the accommodation box 605 supplies power to or charges the radiation imaging apparatus 600, or an imaging operation, the exchange of image information, or the like is performed between the radiation imaging apparatus 600 and the medical cart 604.

As described above, since the power reception connector 63 and the power feeding connector 72 are connected together on the supporting surface 73 on which the thick portion 61 of the radiation imaging apparatus 600 is supported, the risk of a contact failure due to a positional shift between the power reception connector 63 and the power feeding connector 72 is reduced. On the other hand, a case is considered where unlike the configuration of the present embodiment, for example, the radiation imaging apparatus 600 is supported against the bottom surface 74 of the accommodation unit 71 illustrated in FIG. 22A. At this time, the distance from the bottom surface 74 of the accommodation unit 71 to the power feeding connector 72 in the thick portion 61 of the radiation imaging apparatus 600 is long, and therefore, the dimensional tolerance is large. Thus, it is difficult to adjust the positions of the power reception connector 63 and the power feeding connector 72.

To accurately connect the connectors, the dimensional accuracy is extremely important. Thus, the configuration in which the supporting surface 73 that the thick portion 61 contacts is provided in the accommodation unit 71 as in the present embodiment is desirable. Also in terms of the above strengths and the above position adjustment, it is important that the power reception connector 63 is disposed in the thick portion 61, and the supporting surface 73 is provided in the accommodation unit 71.

According to the present embodiment, an accommodation box is achieved in which, when a radiation imaging apparatus is accommodated in the accommodation box, it is possible to prevent the breakage of the radiation imaging apparatus by softening a shock given to the radiation imaging apparatus, and it is also possible to receive and feed power with high reliability by certainly connecting the power reception connector 63 to the power feeding connector 72.

In the present embodiment, a power reception/feeding system includes the radiation imaging apparatus 600 and the accommodation box 605 of the medical cart 603 or 604. In the power reception/feeding system, as described above, when the radiation imaging apparatus 600 is put into the accommodation box 605, power is fed to the radiation imaging apparatus 600. Specifically, on the supporting surface 73 on which the thick portion 61 is supported, the power reception connector 63 as a first power reception/feeding unit of the radiation imaging apparatus 600 and the power feeding connector 72 as a second power reception/feeding unit of the accommodation box 605 are electrically connected together, and power is received and fed between the power reception connector 63 and the power feeding connector 72. As the reception and feeding of power, the medical cart 603 or 604 supplies power to or charges the radiation imaging apparatus 600, or an imaging operation, the exchange of image information, or the like is performed between the radiation imaging apparatus 600 and the medical cart 603 or 604.

According to the present embodiment, a power reception/feeding system is achieved in which, when a radiation imaging apparatus is accommodated in an accommodation member, it is possible to prevent the breakage of the radiation imaging apparatus by softening a shock received by the radiation imaging apparatus, and it is also possible to receive and feed power by certainly connecting the power reception connector 63 to the power feeding connector 72.

### Variation of Sixth Embodiment

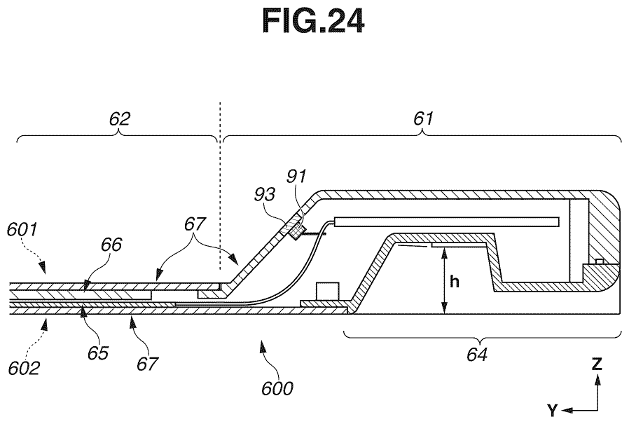

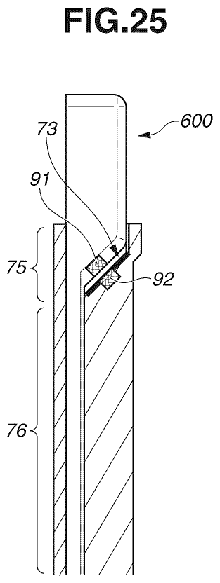

A variation of the sixth embodiment is described. FIG. 24 is a cross-sectional view illustrating a radiation imaging apparatus according to the variation of the present embodiment and corresponds to FIG. 18 in the present embodiment. FIG. 25 is a cross-sectional view illustrating a medical cart including an accommodation box for the radiation imaging apparatus according to the variation of the present embodiment and corresponds to FIG. 22B in the present embodiment.

In the present variation, as illustrated in FIG. 24, in the radiation imaging apparatus 600, as a power reception/feeding unit, a wireless power reception unit 91 capable of wirelessly receiving and feeding power from and to outside is disposed in the thick portion 61 instead of the power reception connector 63 capable of receiving power from outside and feeding the power to the radiation detector. The wireless power reception unit 91 includes a wireless coil that transmits and receives electromagnetic wave energy. The wireless power reception unit 91 is disposed inside the housing 67 in the thick portion 61. In a case where the housing 67 is formed of a metal material such as a magnesium alloy or the like, an opening is formed in a portion of the housing 67 where the wireless power reception unit 91 is disposed. Then, an insulating film 93 of a resin or the like that closes the opening is provided.

In the present variation, the wireless power reception unit 91 is disposed in the sloping portion as the boundary part of the thick portion 61 with the thin portion 62. Although, for example, the wireless power reception unit 91 is disposed in an approximately central part of the sloping portion of the thick portion 61, for example, in view of usability, the wireless power reception unit 91 may be disposed at a position shifted from the central part of the sloping portion to avoid overlap with the gripping portion 64 on the back surface 602 in a plan view. In the radiation imaging apparatus 600 according to the present variation, other components are similar to those of the radiation imaging apparatus 600 according to the present embodiment.

As illustrated in FIG. 25, a wireless power feeding unit 92 capable of wirelessly receiving and feeding power from and to outside is disposed in the supporting surface 73 of the accommodation unit 71 of the medical cart 603 or 604 instead of a power reception/feeding unit such as the power feeding connector 72 capable of, for example, charging the radiation imaging apparatus 600 when the radiation imaging apparatus 600 is attached. The wireless power feeding unit 92 includes a wireless coil that transmits and receives electromagnetic wave energy. In the supporting member according to the present variation, other components are similar to those of the supporting member according to the present embodiment.

When the radiation imaging apparatus 600 is put into the accommodation unit 71 of the medical cart 603 or 604, the sloping portion of the thick portion 61 of the radiation imaging apparatus 600 abuts the supporting surface 73 of the accommodation unit 71, and the radiation imaging apparatus 600 is supported in the accommodation unit 71. In the abutment, the wireless coil of the wireless power reception unit 91 disposed in the sloping portion of the thick portion 61 and the wireless coil of the wireless power feeding unit 92 disposed in the supporting surface 73 of the accommodation unit 71 are opposed to each other and come close to or into contact with each other, and the wireless coils are coupled together by a magnetic field. Consequently, power is received and fed between the accommodation box 605 and the radiation imaging apparatus 600. As the reception and feeding of power, the accommodation box 605 supplies power to or charges the radiation imaging apparatus 600, or an imaging operation, the exchange of image information, or the like is performed between the radiation imaging apparatus 600 and the medical cart 603 or 604.

In the present variation, since power is wirelessly received and fed between the wireless power reception unit 91 and the wireless power feeding unit 92, it is possible to receive and feed power in a somewhat wide region. Thus, even in a case where a slight positional shift occurs between the wireless power reception unit 91 and the wireless power feeding unit 92 in the supporting surface 73, it is possible to certainly feed power. The wireless power reception unit 91 is disposed inside the housing 67, and does not require a part exposed from the housing 67 to use a wireless connection. Thus, when the wireless power reception unit 91 is electrically connected to the wireless power feeding unit 92, or even when the radiation imaging apparatus 600 is dropped, breakage or the like is less likely to occur.

### Seventh Embodiment

In a seventh embodiment, the following describes other examples of the radiation imaging apparatus, the accommodation member for the radiation imaging apparatus, and the power reception/feeding system of the radiation imaging apparatus to which the present disclosure is applied.

### [Radiation Imaging Apparatus According to Present Embodiment]

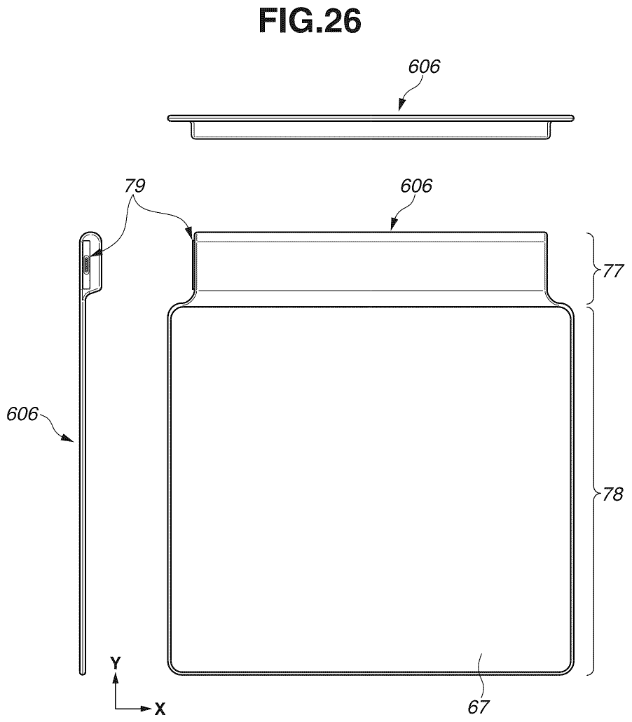

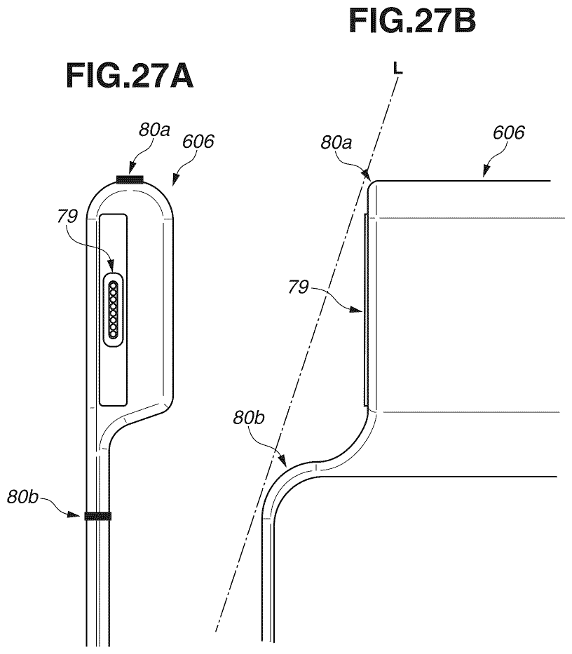

FIG. 26 is a schematic view illustrating a radiation imaging apparatus according to the present embodiment. A central portion shows a plan view, a left portion shows a side view viewed from an X-direction, and an upper portion shows a side view viewed from a Y-direction. FIGS. 27A and 27B are enlarged schematic views of a peripheral portion of a power reception connector of the radiation imaging apparatus. FIG. 27A is a side view viewed from the X-direction in FIG. 26, and FIG. 27B is a plan view.

In a radiation imaging apparatus 606, similarly to the sixth embodiment, the housing 67 includes a thick portion 77 and a thin portion 78 integrated together in the internal space. The radiation detector is contained in the internal space of the housing 67. The portion of the circuit substrate 68 and the control substrate 69 is disposed in the internal space of the thick portion 77, and the portion of the radiation detection panel 65 is disposed in the internal space of the thin portion 78.

In the present embodiment, the thick portion 77 is formed such that the width along the longitudinal direction (the width along the X-direction) of the thick portion 77 is narrower than the width along the X-direction of the thin portion 78. In the radiation imaging apparatus 606, a side surface along the short direction (the Y-direction) of the thick portion 77 is inside the outermost shape of the radiation imaging apparatus 606, and a power reception connector 79 is disposed on the side surface. Each of apex portions of the thick portion 77, apex portions of the thin portion 78, and a connection portion (a sloping portion) of the thick portion 77 with the thin portion 78 may not have an edge shape, and for example, may have a curved surface shape (a rounded shape) as illustrated in FIG. 27B.

The width along the X-direction of the thick portion 77 is comparable to the width of the effective imaging region (not illustrated) of the radiation detection panel 65. In the present embodiment, since the driving substrate 70 is spread without being folded on the radiation detection panel 65, the width in the X-direction from the effective imaging region to the surface of the housing 67 in the thin portion 78 expands to 20 mm or more, for example. The width of the thick portion 77 is set to be comparable to the width of the effective imaging region, whereby it is easy for the user to recognize the effective imaging region using the position of the side surface of the thick portion 77 as a guide. Particularly, this configuration is effective when the user makes rounds or the like and performs work where an indicator provided on an irradiation surface is hard to see.

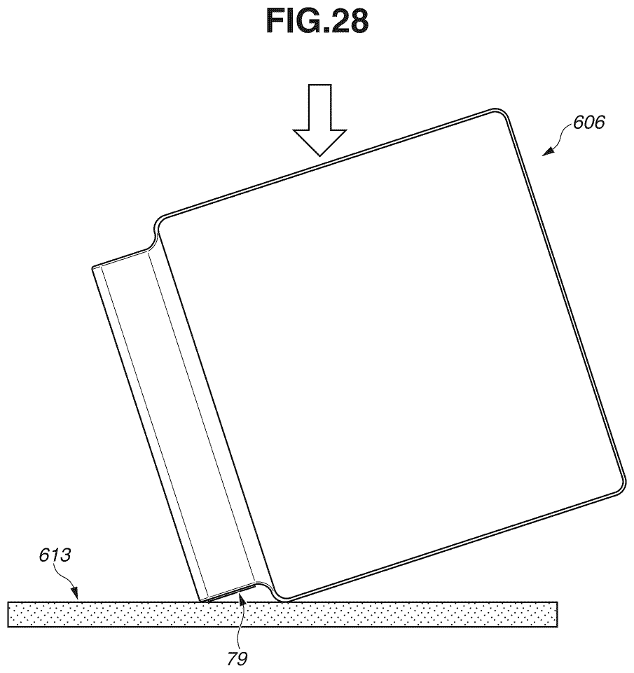

As illustrated in FIGS. 27A and 27B, in the outermost shape of the radiation imaging apparatus 606, the thick portion 77 has an apex 80a, and the thin portion 78 has an apex 80b close to the apex 80a. The power reception connector 79 is disposed in such a manner that the power reception connector 79 is disposed on the inner side (on the radiation imaging apparatus 606 side) of a virtual straight line L indicated by a dashed-dotted line in FIG. 27B connecting the apexes 80a and 80b. When the radiation imaging apparatus 606 is carried around, and even if the radiation imaging apparatus 606 is inadvertently dropped, as illustrated in FIG. 28, the power reception connector 79 does not come into direct contact with the floor 613. Thus, it is possible to reduce the risk of the breakage of the power reception connector 79. The thick portion 77 is heavy compared to the thin portion 78 and therefore is likely to fall from the thick portion 77 when the radiation imaging apparatus 606 is dropped, and the risk of the breakage of the power reception connector 79 installed in the thick portion 77 is high. Thus, the above configuration is effective for the prevention of the breakage of the power reception connector 79. Thus, the power reception connector 79 is disposed on the inner side of the virtual straight line L illustrated in FIG. 27B, whereby, even if the radiation imaging apparatus 606 is dropped, it is possible to prevent the breakage of the power reception connector 79.

It is also possible that components likely to be broken or cause an erroneous operation due to a shock, such as various switches and connectors, are disposed together with the power reception connector 79 (or instead of the power reception connector 79) on the above side surface of the thick portion 77. Also in this case, even if the radiation imaging apparatus 606 is dropped, similarly to the above, it is possible to prevent the breakage of these components.

According to the present embodiment, a radiation imaging apparatus is achieved in which, even if the radiation imaging apparatus receives a shock when the radiation imaging apparatus is carried around or the like, breakage is less likely to occur, and it is possible to easily and certainly receive and feed power with high reliability by electrically connecting the power reception connector 79 disposed in the thick portion 77 of the housing 67 to an external power feeding connector.

### [Supporting Member According to Present Embodiment]

As an accommodation unit for accommodating a radiation imaging apparatus, it is desirable to use a common accommodation unit that can accommodate both the conventional radiation imaging apparatus having a uniform thickness and a standard size and the radiation imaging apparatus according to the present embodiment, because it is advantageous in terms of the size and the number of components. However, even if two types of radiation imaging apparatuses can be put into and taken out of an accommodation unit by simply making the accommodation unit large, the radiation imaging apparatuses cannot be fixed or connected to a power feeding connector. In an accommodation box 608 according to the present embodiment, the above two types of radiation imaging apparatuses can be used in a single accommodation box 608. Specifically, a single accommodation unit includes accommodation regions having different widths in the thickness direction of the accommodation unit, whereby the radiation imaging apparatuses can be used in common.

On the other hand, a power feeding connector capable of charging is provided in the accommodation unit. It is desirable that a single power feeding connector be able to connect in common to the above two types of radiation imaging apparatuses. Accordingly, in an accommodation box according to the present embodiment, a power feeding connector is disposed in a region having a thickness less than or equal to a thickness of the conventional radiation imaging apparatus from a back surface of the accommodation unit, whereby it is possible to position the power feeding connector with power reception connectors of the above two types of radiation imaging apparatuses.

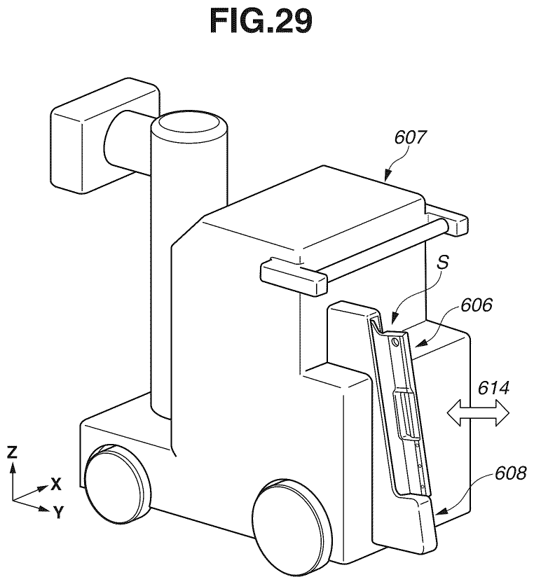

FIG. 29 is a perspective view illustrating a medical cart including the accommodation box for the radiation imaging apparatus according to the present embodiment.

In a medical cart 607, an accommodation box 608 for the radiation imaging apparatus is provided, and the radiation imaging apparatus is put into and taken out of the accommodation box 608 in the direction of an arrow 614.

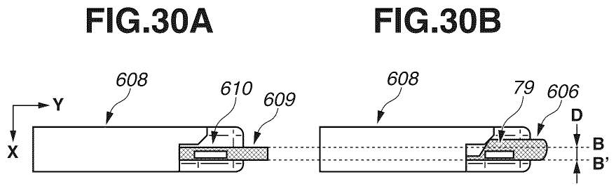

FIGS. 30A and 30B are partial cutaway side views illustrating a portion of the accommodation box 608 when viewed in a direction S in FIG. 29. FIG. 30A illustrates the state where the conventional radiation imaging apparatus is accommodated. FIG. 30B illustrates the state where the radiation imaging apparatus according to the present embodiment is accommodated.

On a side surface of a conventional radiation imaging apparatus 609, a power reception connector 610 is disposed. On the side surface of the thick portion 77 of the radiation imaging apparatus 606 according to the present embodiment, the power reception connector 79 is disposed as described above. When the radiation imaging apparatus 606 or 609 is put into an accommodation unit of the accommodation box 608 according to the present embodiment, the power reception connector 610 or 79 is located at the similar position in the accommodation unit, in this case, at the similar position in a range indicated by a distance D from a back surface B to a front surface B′ of the accommodation unit. The distance D is equivalent to the thickness of the conventional radiation imaging apparatus 609, and for example, has a value in the range of about 15 mm to about 16 mm. As illustrated in FIG. 30A, when the conventional radiation imaging apparatus 609 is put into the accommodation unit of the accommodation box 608, the power reception connector 610 provided on the side surface faces and is electrically connected to a power feeding connector disposed in the accommodation box 608. As illustrated in FIG. 30B, when the radiation imaging apparatus 606 according to the present embodiment is put into the accommodation unit of the accommodation box 608, the power reception connector 79 provided on the side surface of the thick portion 77 faces and is electrically connected to the power feeding connector disposed in the accommodation box 608.

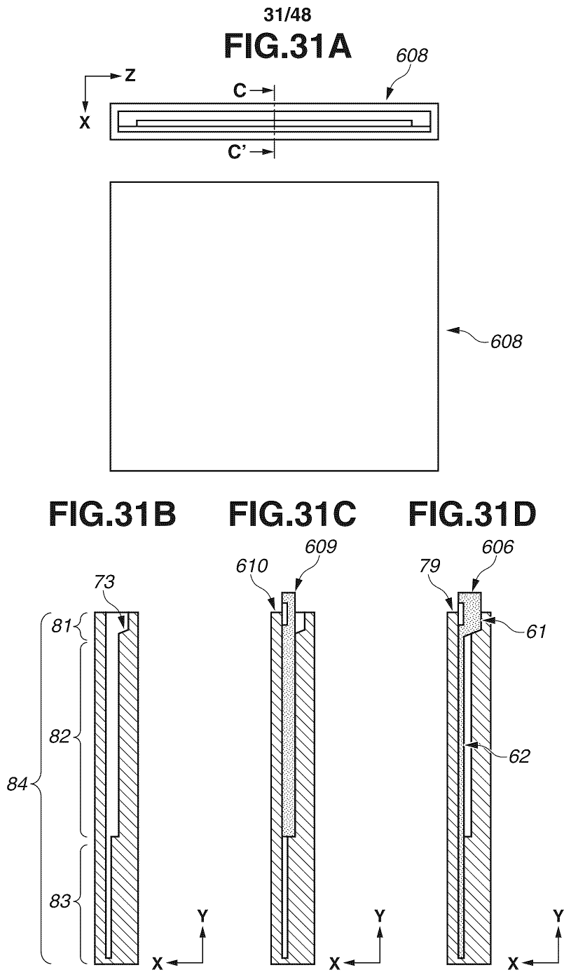

FIGS. 31A to 31D are schematic views illustrating the accommodation box according to the present embodiment. FIG. 31A is a plan view and a side view, and FIGS. 31B to 31D are a cross-sectional view along a dashed-dotted line C-C′ in FIG. 31A. FIG. 31B illustrates the state where nothing is accommodated. FIG. 31C illustrates the state where the conventional radiation imaging apparatus is accommodated. FIG. 31D illustrates the state where the radiation imaging apparatus according to the present embodiment is accommodated.

An accommodation unit 84 includes a thick region 81 as a first region, a thin region 83 as a second region, and a normal region 82 as a third region. The thick region 81 corresponds to the thickness of the thick portion 77 of the radiation imaging apparatus 606 according to the present embodiment and is the widest. The thin region 83 corresponds to the thickness of the thin portion 78 of the radiation imaging apparatus 606 according to the present embodiment and is the narrowest. The normal region 82 corresponds to the thickness of the conventional radiation imaging apparatus 609 and is narrower than the thick region 81 and wider than the thin region 83.

As illustrated in FIG. 31C, the conventional radiation imaging apparatus 609 is put into the accommodation unit 84 of the accommodation box 608. At this time, the radiation imaging apparatus 609 is thicker (about 15 mm to about 16 mm) than the width of the thin region 83, and therefore cannot enter the thin region 83. Then, the radiation imaging apparatus 609 contacts a bottom portion of the normal region 82 and stops. The power reception connector 610 of the radiation imaging apparatus 609 is located at a predetermined place defined based on the back surface B of the accommodation unit 84 as described above and is accurately connected to the power feeding connector of the accommodation box 608.

As illustrated in FIG. 31D, the radiation imaging apparatus 606 according to the present embodiment is put into the accommodation unit 84 of the accommodation box 608. At this time, the radiation imaging apparatus 606 has a thickness corresponding to the width of the thin region 83 and therefore enters the thin region 83. Then, the thick portion 77 contacts the supporting surface 73 of the thick region 81 and stops. The power reception connector 79 of the radiation imaging apparatus 606 is located at a predetermined place defined based on the back surface B of the accommodation unit 84 as described above and is accurately connected to the power feeding connector of the accommodation box 608.

In a case where the radiation imaging apparatus 606 is put into the accommodation unit 84, the radiation imaging apparatus 606 is fixed in the thick portion 77, and therefore, there is a case where the radiation imaging apparatus 606 can be fixed without using the thin region 83, depending on the size of the radiation imaging apparatus 606. However, since in the radiation imaging apparatus 606, the radiation detector has a configuration in which the driving substrate 70 is spread without being folded on the radiation detection panel 65, the outer shape of the thin portion 78 is larger than the outer shape of the conventional radiation imaging apparatus 609. Thus, it is desirable to provide the thin region 83 in the accommodation unit 84. The thin region 83 is brought close to the thickness of the thin portion 78, whereby, when the radiation imaging apparatus 606 is inserted into the accommodation unit 84, the radiation imaging apparatus 606 is not inserted in the state where the radiation imaging apparatus 606 is tilted in the thickness direction. Thus, this configuration also has the advantage that it is possible to prevent the occurrence of a defect such as a so-called half-inserted state or the like.

According to the present embodiment, a highly reliable accommodation box is achieved in which, when a radiation imaging apparatus is accommodated in the accommodation box, it is possible to prevent the breakage of the radiation imaging apparatus by softening a shock given to the radiation imaging apparatus, and it is also possible to certainly connect the power reception connector 79 to the power feeding connector.

In the present embodiment, a power reception/feeding system includes the radiation imaging apparatus 609 and the accommodation box 608 of the medical cart 607. In the power reception/feeding system, as described above, when the radiation imaging apparatus 606 is put into the accommodation box 608, power is fed to the radiation imaging apparatus 606. Specifically, on the supporting surface 73 on which the thick portion 61 is supported, the power reception connector 79 as a first power reception/feeding unit of the radiation imaging apparatus 606 and the power feeding connector as a second power reception/feeding unit of the accommodation box 608 are electrically connected together, and power is received and fed between the power reception connector 79 and the power feeding connector. As the reception and feeding of power, the medical cart 607 supplies power to or charges the radiation imaging apparatus 606, or an imaging operation, the exchange of image information, or the like is performed between the radiation imaging apparatus 606 and the medical cart 607.

According to the present embodiment, a power reception/feeding system is achieved in which, when a radiation imaging apparatus is accommodated in an accommodation member, it is possible to prevent the breakage of the radiation imaging apparatus by softening a shock received by the radiation imaging apparatus, and it is also possible to receive and feed power by certainly connecting the power reception connector 79 to the power feeding connector.

Also in the present embodiment, similarly to the variation of the sixth embodiment, a wireless power reception unit may be disposed instead of the power reception connector 79 of the radiation imaging apparatus 606, and a wireless power feeding unit may be disposed instead of the power feeding connector of the accommodation box 608.

### Eighth Embodiment

In an eighth embodiment, the following describes another example of the radiation imaging apparatus to which the present disclosure is applied.

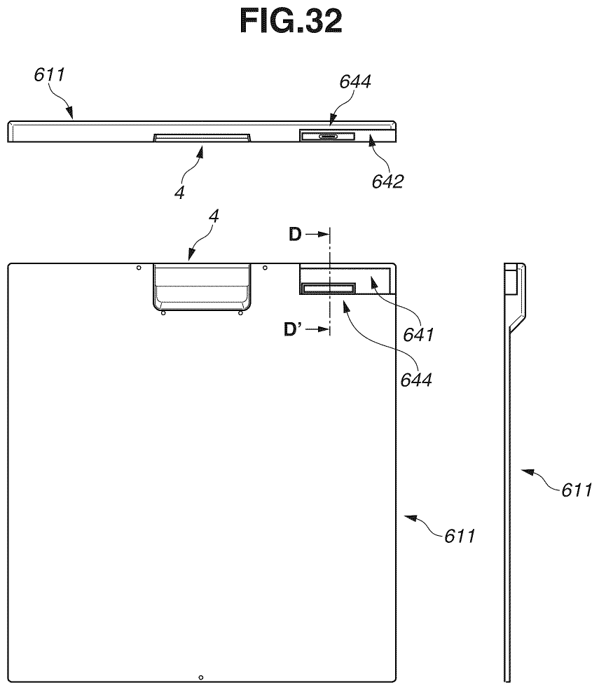

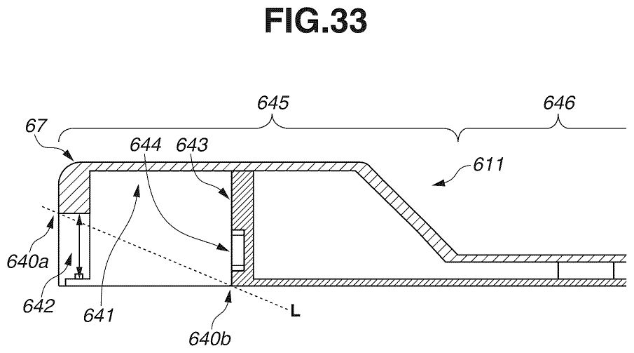

FIG. 32 is a schematic view illustrating a radiation imaging apparatus according to the present embodiment. A central portion shows a plan view, a right portion shows a right side view, and an upper portion shows an upper side view. FIG. 33 is a cross-sectional view along a dashed-dotted line D-D′ in FIG. 32.

In a radiation imaging apparatus 611, similarly to the sixth embodiment, the housing 67 includes a thick portion 645 and a thin portion 646 integrated together in the internal space. The radiation detector is contained in the internal space of the housing 67. The portion of the circuit substrate 68 and the control substrate 69 is disposed in the internal space of the thick portion 645, and the portion of the radiation detection panel 65 is disposed in the internal space of the thin portion 646.

In the present embodiment, in an end portion of a back surface in the thick portion 645 of the radiation imaging apparatus 611, a depression 641 is provided. At the outermost shape part of the depression 641, a step 642 between the depression 641 and a back surface of the housing 67 is formed. On a side wall surface 643 of the depression 641, a power reception connector 644 is disposed. The radiation imaging apparatus 611 has an apex 640a on an outermost surface of the step 642 and an apex 640b on the side wall surface 643. The power reception connector 644 is disposed at a position on the inner side (on the radiation imaging apparatus 611 side) of a virtual straight line L indicated by a dashed line in FIG. 33 connecting the apexes 640a and 640b.

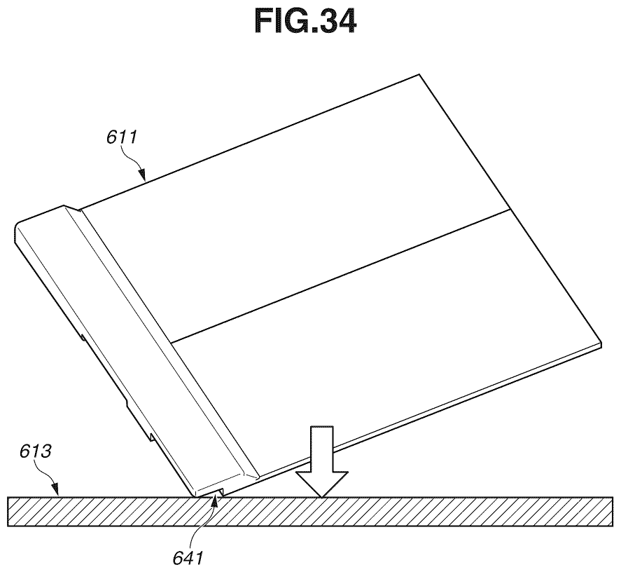

When the radiation imaging apparatus 611 is carried around, and even if the radiation imaging apparatus 611 is inadvertently dropped, as illustrated in FIG. 34, the power reception connector 644 does not come into direct contact with the floor 613 because of the step 642. Thus, it is possible to reduce the risk of the breakage of the power reception connector 644. The thick portion 645 is heavy compared to the thin portion 646 and therefore is likely to fall from the thick portion 645 when the radiation imaging apparatus 611 is dropped, and the risk of the breakage of the power reception connector 644 installed in the thick portion 645 is high. Thus, the above configuration is effective for the prevention of the breakage of the power reception connector 644. Thus, the power reception connector 644 is disposed on the inner side of the virtual straight line L illustrated in FIG. 33, whereby, even if the radiation imaging apparatus 611 is dropped, it is possible to prevent the breakage of the power reception connector 644.

Also in the present embodiment, similarly to the sixth embodiment, an accommodation box including a power feeding connector to be electrically connected to the power reception connector 644 of the radiation imaging apparatus 611 when the radiation imaging apparatus 611 is accommodated is provided. A power reception/feeding system including the radiation imaging apparatus 611 and the accommodation box is provided.

Also in the present embodiment, similarly to the variation of the seventh embodiment, a wireless power reception unit may be disposed instead of the power reception connector 644 of the radiation imaging apparatus 611, and a wireless power feeding unit may be disposed instead of the power feeding connector of the accommodation box.

One of the radiation imaging apparatuses according to the sixth embodiment, the variations of the sixth embodiment, the seventh embodiment, and the eighth embodiment can also be applied to the radiation imaging apparatus 1601 of the radiation imaging system described with reference to FIG. 16 in the other embodiments.

Basic Configuration of Power Transmission/Reception Apparatus to which Present Disclosure is Applied

Next, ninth and tenth embodiments are described. To describe the ninth and tenth embodiments, the following describes the basic configuration of a power transmission/reception apparatus to which the present disclosure is applied.

The power transmission/reception apparatus according to the present disclosure can accommodate a radiation imaging apparatus including a first wireless power transmission/reception unit that wirelessly transmits and receives power to and from outside. The radiation imaging apparatus includes a radiation detection panel that converts radiation transmitted through an object into an electric signal. The power transmission/reception apparatus includes a second wireless power transmission/reception unit that wirelessly transmits and receives power to and from the first wireless power transmission/reception unit, and an adjustment mechanism as a position adjustment unit that, in a case where the radiation imaging apparatus is accommodated, adjusts a position of the radiation imaging apparatus so that the first and second wireless power transmission/reception units come close to each other and have a positional relationship where the power transmission/reception apparatus can wirelessly feed power to the radiation imaging apparatus. The first and second wireless power transmission/reception units include wireless coils for transmitting and receiving electromagnetic wave energy, and the wireless coils are opposed to each other and come close to each other, and are coupled together by an electromagnetic field. Consequently, power is transmitted and received between the radiation imaging apparatus and the power transmission/reception apparatus. There are a variety of radiation imaging apparatuses different in thickness or the like from each other, and there is a case where, when the radiation imaging apparatus is accommodated in the power transmission/reception apparatus, the first and second wireless power transmission/reception units are separate from each other. Even in such a case, the radiation imaging apparatus moves by the adjustment mechanism, and the first wireless power transmission/reception unit comes close to the second wireless power transmission/reception unit. Consequently, it is possible to wirelessly transmit and receive power for charging or the like stably and efficiently.

In the radiation imaging apparatus and the power transmission/reception apparatus, the power transmission/reception apparatus can supply power to or charge the radiation imaging apparatus, or the apparatuses can perform an imaging operation or exchange image information via the second wireless power transmission/reception unit and the first wireless power transmission/reception unit. As the power transmission/reception apparatus according to the present disclosure, a charging apparatus that mainly charges the radiation imaging apparatus is described. In embodiments described below, as an example, a case is illustrated where the first wireless power transmission/reception unit is a wireless power reception unit that receives power for charging, and the second wireless power transmission/reception unit is a wireless power transmission unit that transmits power for charging.

For example, the adjustment mechanism includes a main surface pressing member. The main surface pressing member presses a main surface of the accommodated radiation imaging apparatus in a thickness direction of the radiation imaging apparatus so that the first wireless power transmission/reception unit comes close to the second wireless power transmission/reception unit. For example, the adjustment mechanism includes a side surface pressing member. The side surface pressing member presses a side surface of the accommodated radiation imaging apparatus in a direction intersecting a thickness direction of the radiation imaging apparatus so that the first wireless power transmission/reception unit comes close to the second wireless power transmission/reception unit. The main surface pressing member or the side surface pressing member brings the first wireless power transmission/reception unit close to the second wireless power transmission/reception unit, and it is possible to efficiently transmit and receive power for charging or the like.

For example, the adjustment mechanism includes an inclined roller member. The inclined roller member is disposed at a place that receives a self-weight of the accommodated radiation imaging apparatus, and the inclined roller member adjusts the position of the accommodated radiation imaging apparatus by rotation of the inclined roller member so that the first wireless power transmission/reception unit comes close to the second wireless power transmission/reception unit. The inclined roller member is provided, whereby it is possible to easily and securely bring the first wireless power transmission/reception unit of the accommodated radiation imaging apparatus close to the second wireless power transmission/reception unit using gravity without providing a complex and large driving apparatus or the like.

The power transmission/reception apparatus includes an accommodation unit including a slot in which the radiation imaging apparatus is accommodated. In the accommodation unit, among surfaces forming the slot, at least one surface that receives a self-weight of the accommodated radiation imaging apparatus is inclined from horizontal or vertical. Specifically, a bottom surface forming the slot is inclined with respect to the horizontal so that one side in a short direction of the slot is lower than the other side in the short direction opposed to the one side. Consequently, the radiation imaging apparatus inserted into the slot slides in contact with the bottom surface toward the one side in the short direction by the self-weight of the radiation imaging apparatus, and the first wireless power transmission/reception unit comes close to the second wireless power transmission/reception unit. In another specific example, a bottom surface and at least one of main surfaces forming the slot are inclined from horizontal and vertical so that one side in a longitudinal direction of the slot is lower than the other side in the longitudinal direction opposed to the one side, while the bottom surface and the at least one main surface keep perpendicular to each other. Consequently, when the radiation imaging apparatus is inserted into the slot, the first wireless power transmission/reception unit is disposed in the proximity of the second wireless power transmission/reception unit.

The power transmission/reception apparatus according to the present disclosure can accommodate radiation imaging apparatuses different in thickness or the like from each other, and the adjustment mechanism brings the first and second wireless power transmission/reception units close to each other, regardless of the thickness or the like of a radiation imaging apparatus. As described above, the power transmission/reception apparatus can accommodate a radiation imaging apparatus outside a conventional standard, and automatically brings the first wireless power transmission/reception unit close to the second wireless power transmission/reception unit in a variety of accommodated radiation imaging apparatuses.

As specific examples of the radiation imaging apparatuses that can be accommodated in the power transmission/reception apparatus, the following radiation imaging apparatuses are described.
(1) A radiation imaging apparatus including a thin portion partially or entirely overlapping an effective imaging region when viewed in a thickness direction of the apparatus, and a thick portion thicker than the thin portion. The first wireless power transmission/reception unit is disposed in the thin portion, and a control unit that controls the first wireless power transmission/reception unit is disposed in the thick portion.
(2) A radiation imaging apparatus to which a cover is attached. As various covers, at least one type selected from a grid cover that reduces predetermined radiation, an imaging assistance cover that adjusts a contact state between the apparatus and an object, a protective cover that protects the apparatus from contamination and water immersion, and the like is possible.

As the forms of the power transmission/reception apparatus according to the present disclosure, for example, a first form and a second form described below are possible. The first form will be described in detail below in the ninth embodiment, and the second form will be described in detail below in the tenth embodiment.

### (1) First Form

The power transmission/reception apparatus includes an accommodation unit including a slot. The first wireless power transmission/reception unit is disposed on a first main surface of the radiation imaging apparatus, and the second wireless power transmission/reception unit is disposed on a second main surface of the slot. In a case where the radiation imaging apparatus is accommodated in the accommodation unit, the radiation imaging apparatus moves in friction contact with a bottom surface of the slot by a self-weight of the radiation imaging apparatus, and the first wireless power transmission/reception unit faces the second wireless power transmission/reception unit. The adjustment mechanism adjusts a position of the radiation imaging apparatus to press the first main surface toward the second main surface in a state where the first and second wireless power transmission/reception units are opposed to each other.

### (2) Second Form

The power transmission/reception apparatus includes an accommodation unit including a slot. The first wireless power transmission/reception unit is disposed on a first main surface of the radiation imaging apparatus. The second wireless power transmission/reception unit is disposed on a second main surface of the slot. In a case where the radiation imaging apparatus is accommodated in the accommodation unit, the first wireless power transmission/reception unit faces the second wireless power transmission/reception unit. The adjustment mechanism adjusts a position of the radiation imaging apparatus to press the first main surface toward the second main surface in a state where the first and second wireless power transmission/reception units are opposed to each other.

In the first and second forms, specifically, the first main surface is a surface on a back side when viewed in a thickness direction of the radiation imaging apparatus among main surfaces of the radiation imaging apparatus. It is desirable that the adjustment mechanism press the first main surface toward the second main surface so that an opposing distance in a thickness direction of the radiation imaging apparatus between the first and second wireless power transmission/reception units is 5 mm or less.

With reference to the drawings, suitable embodiments to which the present disclosure can be applied are described in detail below. As a power transmission/reception apparatus according to each of the embodiments, a charging apparatus that wirelessly charges a cartridge as a radiation imaging apparatus is illustrated as an example.

### Ninth Embodiment

### (Cartridge 700)

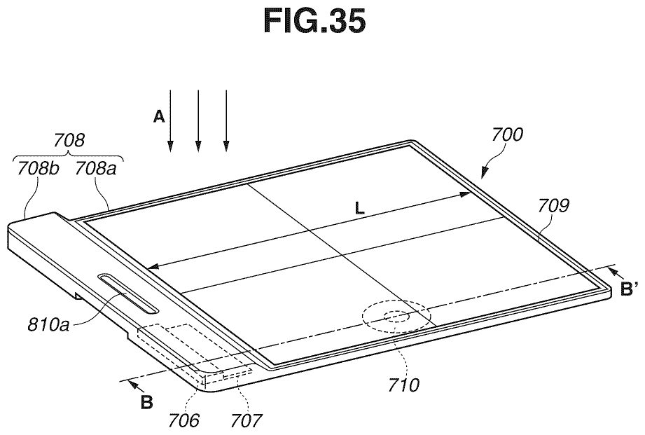

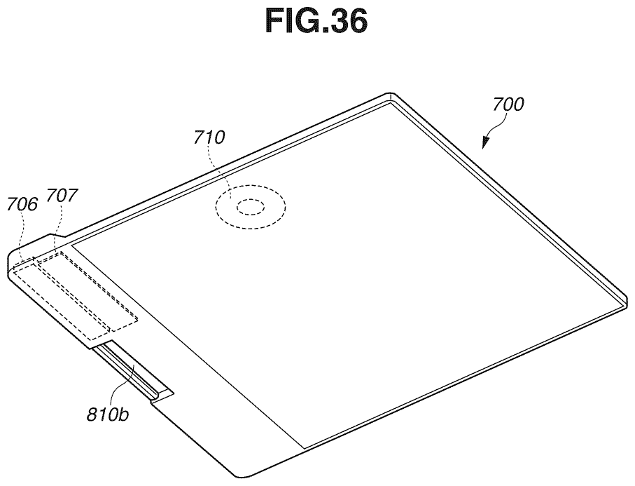

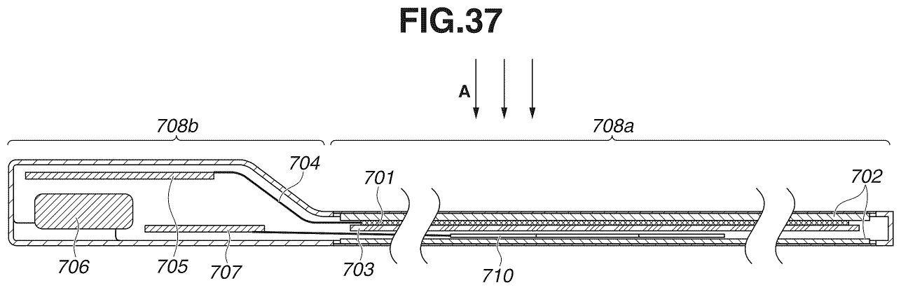

With reference to FIGS. 35 to 48, a cartridge 700 according to the ninth embodiment is described. FIG. 35 is a top perspective view of the cartridge 700 according to the ninth embodiment. FIG. 36 is a bottom perspective view of the cartridge 700 according to the ninth embodiment. FIG. 37 is a cross-sectional view along the direction of a dashed-dotted line B-B′ in FIG. 35 and partially omits intermediate portions. Although among various cartridges that can be used in combination with a charging apparatus according to the present embodiment, the cartridge 700 in a characteristic form is described, a conventional flat plate-like cartridge 711 having an approximately uniform thickness can also be applied to the charging apparatus. The flat plate-like cartridge 711 will be described below.

The cartridge 700 generates a radiation image according to radiation emitted from a radiation generating apparatus (not illustrated) and transmitted through an object. The cartridge 700 transfers the generated radiation image to an external apparatus, and the transferred radiation image is displayed on an external display device or the like. The cartridge 700 includes a radiation detection panel 701 that converts radiation into electric signals. The radiation detection panel 701 has the function of converting radiation incident from a side A (hereinafter, the side A is an incident surface side, and a surface opposed to the side A is a back surface side) and indicated by arrows in FIG. 35 into electric signals. The radiation detection panel 701 includes a sensor substrate on which a plurality of photoelectric conversion elements (sensors) is two-dimensionally arranged on a glass substrate, a phosphor layer disposed on the sensor substrate, and a phosphor protection film disposed on the phosphor layer. The phosphor protection film is composed of a material having relatively high moisture proof properties and is used to protect the phosphor layer.

With the above configuration, in the radiation detection panel 701, the phosphor layer emits light by incident radiation, and the photoelectric conversion elements disposed on the sensor substrate convert the emitted light into electric signals. In the radiation detection panel 701, some or all of the photoelectric conversion elements are an effective imaging region. The effective imaging region is a region where radiation imaging can be performed and an image is actually generated. However, the radiation detection panel 701 is not limited to the above configuration. A plurality of phosphor layers may be provided, or direct conversion type conversion elements that directly convert radiation into electric signals may be used instead of the phosphor layer and the photoelectric conversion elements. The material of the sensor substrate of the radiation detection panel 701 is not limited to glass, either, and may be a resin material or the like having a higher flexibility.

The radiation detection panel 701 is electrically connected to a control substrate 705 via a flexible circuit substrate 704. The control substrate 705 reads electric signals into which radiation has been converted by the radiation detection panel 701, and performs various processes. For example, the control substrate 705 converts the electric signals into digital signals, thereby generating radiation image data. The control substrate 705 also transfers the generated radiation image data to an external apparatus.

The cartridge 700 includes a secondary battery 706 that supplies power to the radiation detection panel 701 and the control substrate 705. For example, the secondary battery 706 is a lithium-ion battery, an electrical double layer capacitor, an all-solid-state battery, or the like. The secondary battery 706 may be attachable to and detachable from the cartridge 700, or may be fixed to the cartridge 700.

A wireless power reception unit 710 as a first wireless power transmission/reception unit receives power supplied from a wireless power transmission unit 810 in a charging apparatus 800. The received power is rectified via a charging circuit substrate 707 including a control unit that controls the wireless power reception unit 710, and is used to charge the secondary battery 706 with a predetermined voltage. The cartridge 700 and the charging apparatus 800 transmit and receive power using an electromagnetic induction method as an example of a wireless charging method. Particularly, in the case of the electromagnetic induction method, in a case where the central axes of a power transmission side coil and a power reception side coil come as close to each other as possible and ultimately coincide with each other, and the surfaces of the coils come as close to each other as possible, the charging efficiency becomes high. This acquired accuracy of closeness varies depending on the types of the coils or the surrounding conditions of the coils. In the present embodiment, each of the distance between the central axes and the distance between the surfaces is about 5 mm or less. Although each of the wireless power reception unit 710 according to the present embodiment and the wireless power transmission unit 810 is a single circular coil, the present disclosure is not limited to this. The number or the shapes of coils are appropriately changed according to the arrangement in the cartridge 700 or the charging apparatus 800 or the charging power. For example, a coil having an elliptical or oblong shape may be used. Although the surface direction of the wireless power reception unit 710 is disposed parallel to the surface of the radiation detection panel 701, the surface direction of the wireless power reception unit 710 may be disposed to intersect the surface of the radiation detection panel 701 where necessary. As another wireless charging method, a radio wave reception type or a resonance type may be used.

A supporting base 703 supports the radiation detection panel 701 on an incident surface side of the supporting base 703 and supports the wireless power reception unit 710 on a back surface side of the supporting base 703.

A housing 708 contains the above components. The housing 708 includes a thin portion 708a and a thick portion 708b. The thin portion 708a mainly contains the radiation detection panel 701, the wireless power reception unit 710, and components close to the wireless power reception unit 710. On the other hand, the thick portion 708b mainly contains electric components such as the control substrate 705, the secondary battery 706, the charging circuit substrate 707, and the like. Buffer materials 702 that protect the radiation detection panel 701 from an external force are provided between the thin portion 708a and the radiation detection panel 701. It is desirable that an incident surface side of the thin portion 708a be formed of a material having relatively high radiation transmittance to obtain sharper radiation image data.

As described above, thick components such as the secondary battery 706 and the like are disposed in the thick portion 708b along one side of the radiation detection panel 701, whereby it is possible to make the effective imaging region thinner in comparison with a conventional cartridge as in the thin portion 708a. That is, when imaging is performed, it is possible to reduce the thickness of an insertion portion when the cartridge 700 is inserted between a supine patient and a tabletop, and it is possible to reduce the patient's pain. Simultaneously, it is also possible to reduce the physical burden felt by an imaging operator, such as insertion resistance or the like. When this imaging is performed, the thick portion 708b is mainly disposed approximately parallel to the backbone under the supine patient's arm. Thus, it is desirable that the dimension (indicated by L in FIG. 35) of the thin portion 708a in a direction orthogonal to the thick portion 708b be a sufficient width to include the body width of the patient in an image. For example, a dimension corresponding to a medium size to a large size among conventional cartridge standards, such as 380 mm to 500 mm or the like, can be considered.

Although a case is illustrated where the wireless power reception unit 710 is disposed on the back surface side of the radiation detection panel 701 in the thin portion 708a, the wireless power reception unit 710 may be disposed in the thick portion 708b. Particularly, in the ninth embodiment, no matter which of the thin portion 708a and the thick portion 708b the wireless power reception unit 710 is disposed in, the influence on the charging work of the user does not differ. In the tenth embodiment, however, it is desirable that the wireless power reception unit 710 be disposed in the thin portion 708a. The reason will be described below.

On the surface on the incident surface side of the thin portion 708a, an indicator 709 indicating the effective imaging region and a center point of the effective imaging region is provided, and the user can visually identify the effective imaging region using the indicator 709. For example, the indicator 709 is formed by performing surface treatment such as painting, printing, texturing, blasting, or the like on the thin portion 708a, forming a shape such as a depression or the like on the thin portion 708a, or the combination of these.

On the front and back of an approximately central portion of the thick portion 708b, gripping portions 810a and 810b, respectively, having recessed shapes are formed. The user can stably grip the cartridge 700 by putting their pollex on the gripping portion 810a and hooking other fingers on a step portion of the gripping portion 810b. The gripping portion 810a may not be a single continuous recessed surface as illustrated in FIG. 35, and may have a shape such as a rib, a dot, or the like, or may have a protruding shape. Alternatively, a shape may not be provided, and the gripping properties of the gripping portion 810a may be improved by partially using a material having high frictional properties, such as a rubber material or the like, or using surface treatment such as high-friction painting, texturing, or the like. The above shapes, materials, surface treatment, and the like may be appropriately combined together. The gripping portion 810b may also have various shapes, materials, and the like. The arrangement of the gripping portions 810a and 810b may be reversed. A form may be employed in which the gripping portions 810a and 810b are linked together and penetrate the thick portion 708b.

### (Cartridge 711)

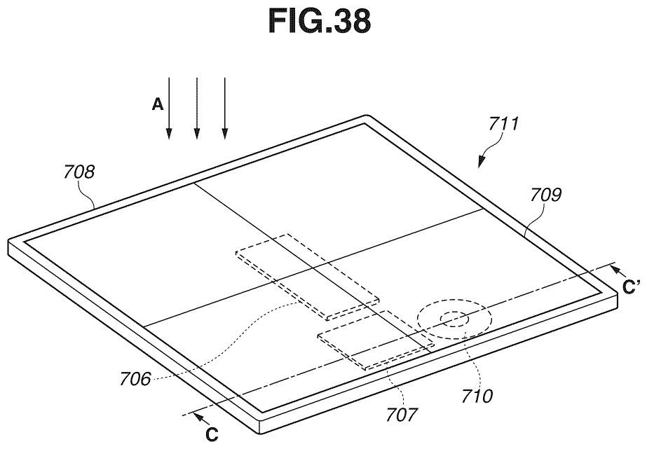

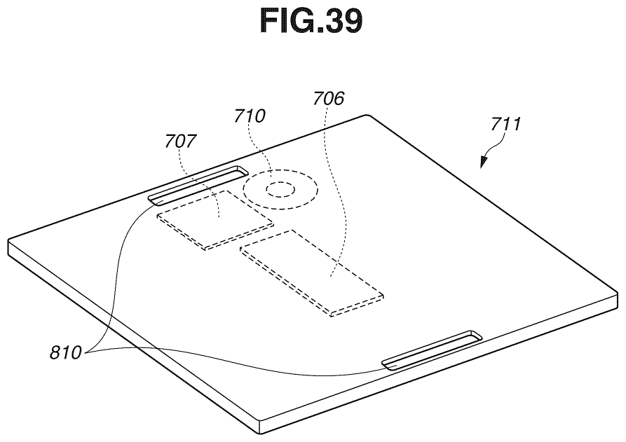

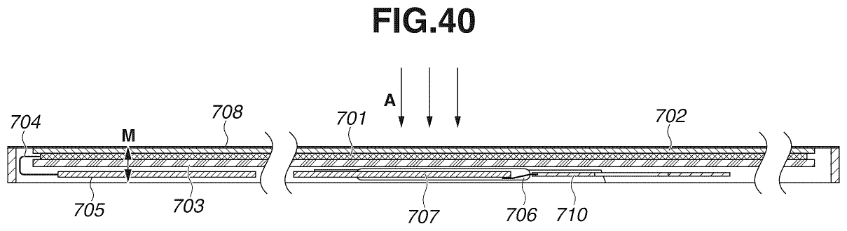

With reference to FIGS. 38 to 40, a cartridge 711 according to the ninth embodiment is described. FIG. 38 is a top perspective view of the cartridge 711 according to the ninth embodiment. FIG. 39 is a bottom perspective view of the cartridge 711 according to the ninth embodiment. FIG. 40 is a cross-sectional view along the direction of a dashed-dotted line C-C′ in FIG. 38 and partially omits intermediate portions. In FIGS. 38 to 40, components similar to those of the cartridge 700 are designated by the same reference numerals as those of the cartridge 700, and are not described.

The cartridge 711 is a conventionally mainstream flat plate-like cartridge having an approximately uniform thickness unlike the cartridge 700 including the thin portion 708a and the thick portion 708b. A basic thickness M of the cartridge 711 may be 13 mm or less, such as 10 mm, 8 mm, or the like, or may be 17 mm or more, such as 20 mm, 30 mm, or the like, in addition to about 15 mm±2 mm according to the standard. On the other hand, the size of a flat surface orthogonal to the thickness direction is about 300 mm to 500 mm on each side.

In the cartridge 711, electric components such as the control substrate 705, the secondary battery 706, the charging circuit substrate 707, and the like are also disposed on the back surface side of the radiation detection panel 701 in addition to the wireless power reception unit 710. A buffer material 702 is provided only on the incident surface side of the radiation detection panel 701, and a gripping portion 810 is provided only on a surface on the back surface side of the housing 708.

### (Charging Apparatus 800)

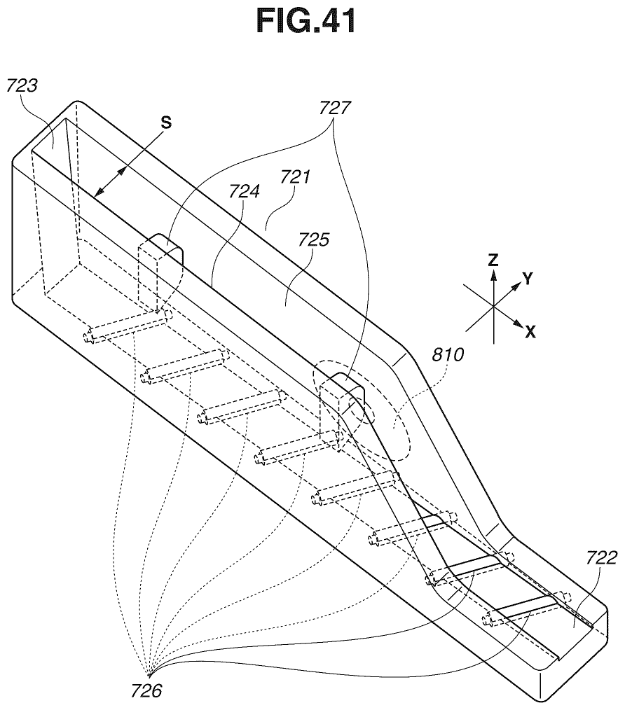

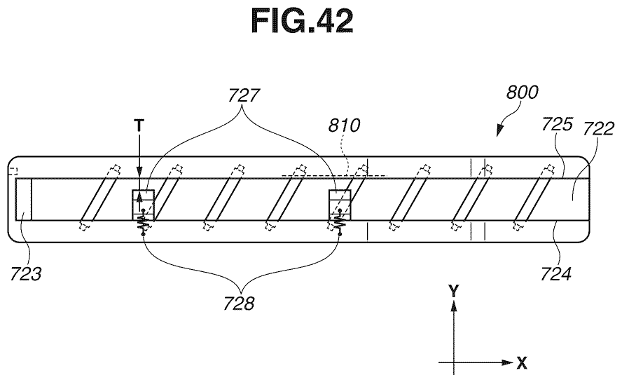

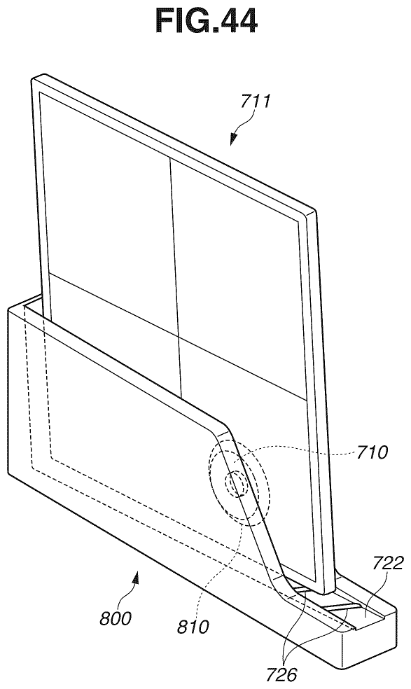

With reference to FIGS. 41 to 44, a charging apparatus 800 according to the ninth embodiment is described. FIG. 41 is a perspective view of the charging apparatus 800 according to the ninth embodiment. FIG. 42 is a top view of the charging apparatus 800 according to the ninth embodiment. FIG. 44 is a perspective view illustrating the state where the cartridge 711 is accommodated in the charging apparatus 800 according to the ninth embodiment. In the charging apparatus 800, an X-direction in FIG. 41 is a width direction, a Y-direction in FIG. 41 is a thickness direction, and a Z-direction in FIG. 41 is a height or up-down direction.

A slot 721 is formed in the charging apparatus 800. The slot 721 is a recessed portion that accommodates cartridges. Hereinafter, “cartridges” is a collective term for a cartridge alone and a cartridge in the state where various covers and the like are attached to the cartridge. A thickness (width) S of the slot 721 is greater than the maximum thickness of the cartridges expected to be accommodated. A bottom surface 722 and a side surface 723 in the slot 721 are inclined to rotate about the Y-axis in FIG. 41 while keeping orthogonal to each other. A front surface 724 (one of main surfaces) and a rear surface 725 (the other main surface) are a side surface on the front side in the thickness direction and a side surface on the rear side in the thickness direction, respectively, in the slot 721.

Inclined rollers 726 as a position adjustment unit for the cartridge 700 or the like are a plurality of rollers provided in a bottom surface portion in the slot 721, and rotation shafts of the inclined rollers 726 are inclined from the Y-axis in a surface parallel to the bottom surface 722. Although in the present embodiment, eight inclined rollers 726 are illustrated as an example, the number of inclined rollers 726 may be any number greater than or equal to two. Main surface pressing members 727 as a position adjustment unit for the cartridge 700 or the like are each provided with a part of the main surface pressing member 727 protruding from the front surface 724 and biased toward the rear surface 725 by a spring 728 (not illustrated in FIG. 41). A gap T between the main surface pressing member 727 and the rear surface 725 when an external force is not applied is less than or equal to the minimum thickness of the cartridges expected to be accommodated. When the cartridges are inserted into the slot 721, and a force in an approximately Y-direction is applied to the main surface pressing member 727, the spring 728 is compressed, and the main surface pressing member 727 is retracted to the position where the gap T is greater than the maximum thickness of the cartridges expected to be accommodated. It is desirable that when the cartridges are inserted, sliding contact portions of the main surface pressing member 727, the side surface 723, and the rear surface 725 that slide in contact with the cartridges each be formed of a material or a surface property having excellent sliding properties. Alternatively, sliding contact properties with the cartridges may be improved by reducing a sliding contact area by providing a rib shape or a dot shape, or providing a rolling member such as a roller, a ball, or the like in a portion that slides in contact with the cartridges. Although in the present embodiment, the main surface pressing members 727 are provided at two places along the width direction, this is to improve the parallelism between the cartridges and the rear surface 725 when the accommodated cartridges are pressed toward the rear surface 725. The number, the sizes, and the shapes of main surface pressing members 727 may be appropriately changed. A configuration may be employed in which the cartridges are pressed using a member having cushioning properties or elasticity, instead of being pressed by springs.

The wireless power transmission unit 810 as a second wireless power transmission/reception unit is provided on the rear surface 725 side and includes a coil for wirelessly transmitting power to the cartridge 700. the following describes behaviors when the cartridge 700 as an example of the cartridges is accommodated in the charging apparatus 800.

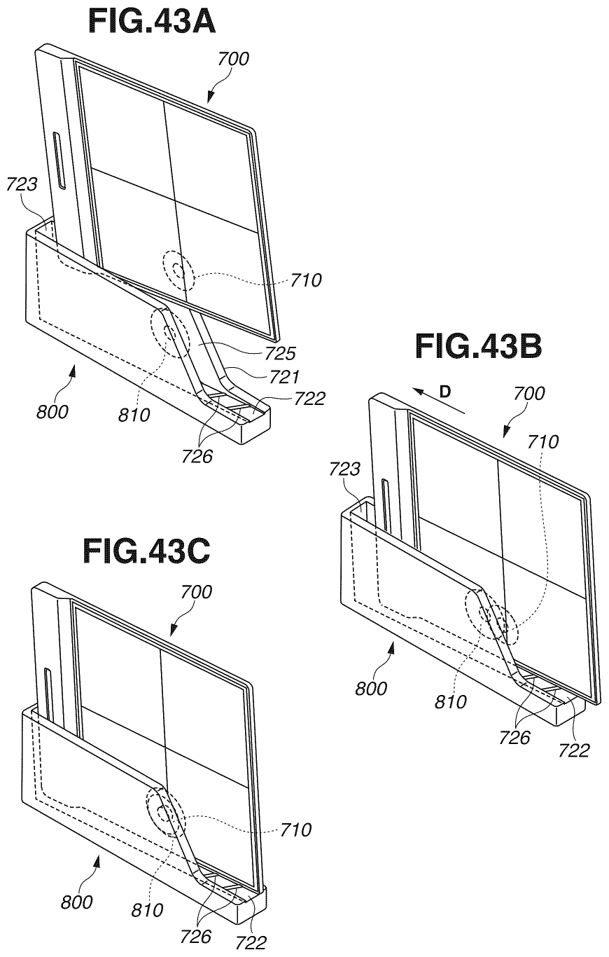

As illustrated in FIG. 43A, when the user accommodates the cartridge 700 in the slot 721 with a back surface side of the cartridge 700 facing the rear surface 725, the cartridge 700 is pressed to the rear surface 725 side by the main surface pressing members 727. Further, when the accommodation proceeds to FIG. 43B, and the cartridge 700 is released from holding by the user and enters the state where the cartridge 700 is on the inclined rollers 726, the cartridge 700 moves by the self-weight of the cartridge 700 in the direction (the direction of an arrow D in FIG. 43B) in which the cartridge 700 contacts the side surface 723 along the inclination of the bottom surface 722. The shafts of the inclined rollers 726 that passively rotate by this motion are inclined, whereby a lower end portion of the cartridge 700 moves to the rear surface 725 side, and the back surface of the cartridge 700 and the rear surface 725 come into further contact with each other. Due to the inclination of the inclined rollers 726 in addition to the pressing by the main surface pressing members 727, the back surface of the cartridge 700 is pressed to the rear surface 725 side more certainly. By the above behaviors, the position of the accommodated cartridge 700 is guided to the state of FIG. 43C where the cartridge 700 contacts the side surface 723 and the rear surface 725, and is held. When the cartridge 700 is in the state of FIG. 43C, the wireless power reception unit 710 and the wireless power transmission unit 810 are disposed in the respective apparatuses so that the distance between the centers of the coils and the distance between the coils in the surface direction are as close to each other as possible, particularly in this case, about 5 mm or less. The cartridge 700 is held in the state of FIG. 43C, i.e., at the position where the wireless power reception unit 710 is pressed toward the wireless power transmission unit 810, whereby it is possible to maintain high wireless charging efficiency.

The above behaviors are also similar in a case where the cartridge 711 is accommodated. FIG. 44 corresponds to the state of FIG. 43C.

A case is also described where the cartridge 711 to which the above various covers are attached is accommodated in the charging apparatus 800. In a case where a cover is attached, the thickness of an object to be accommodated increases. As a cover that particularly greatly increases the thickness, a grid cover, a cushion cover, or the like is considered. All these covers mainly increase the thickness on an incident surface side of the cartridge 711, and an increase in the thickness on a back surface side of the cartridge 711 is small. Specifically, the increase in the thickness on the incident surface side is considered to be a maximum of about +10 mm, and the increase in the thickness on the back surface side is considered to be a maximum of about +1 mm. It is considered that in the above vinyl cover, both the increases in the thicknesses on the incident surface side and the back surface side are a maximum of about +1 mm or less. Thus, similarly to the case of the above cartridge 700, a configuration is employed in which the wireless power reception unit 710 is provided on the back surface side of the cartridge 711, and the cartridge 711 is pressed in the direction in which the back surface of the cartridge 711 and the rear surface 725 come close to each other in the slot 721. Consequently, the wireless power reception unit 710 is pressed toward the wireless power transmission unit 810, and it is possible to maintain high wireless charging efficiency. However, since the increase in the thickness on the back surface side is not zero, either, in view of this, it is desirable that the wireless power reception unit 710 and the wireless power transmission unit 810 be disposed in the proximity of outer surfaces of the respective apparatuses as possible in the apparatuses.

### Tenth Embodiment

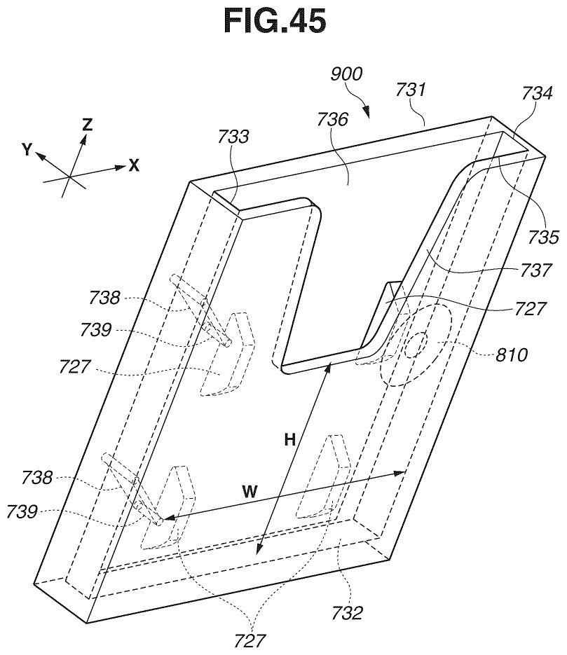

With reference to FIGS. 45 to 48, a charging apparatus according to the tenth embodiment is described. In the following description, components similar to those in the ninth embodiment are designated by the same signs as those in the ninth embodiment, and are not described in detail.

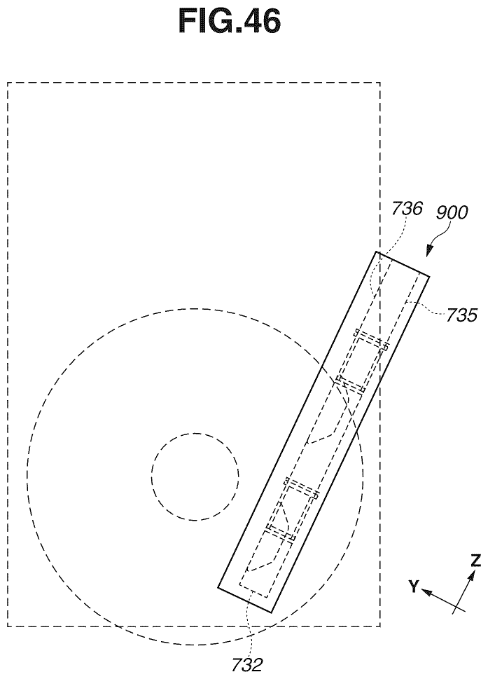

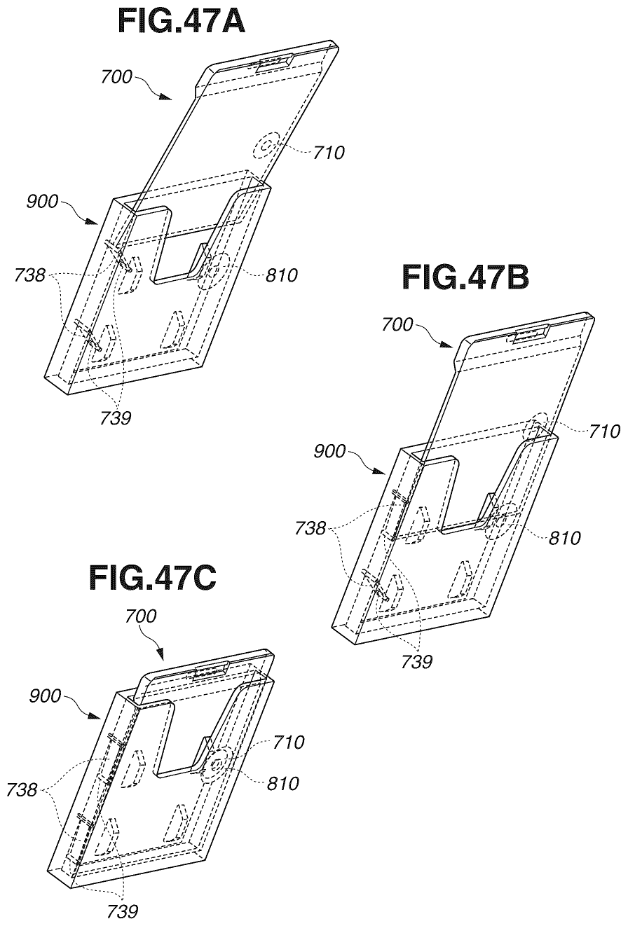

FIG. 45 is a perspective view of a charging apparatus 900 according to the tenth embodiment. FIG. 46 is a side view of the charging apparatus 900 according to the tenth embodiment. FIGS. 47A to 47C are perspective views illustrating a series of behaviors when the cartridge 700 is accommodated in the charging apparatus 900 according to the tenth embodiment. In the present embodiment, a charging apparatus provided integrally with a movable X-ray generating apparatus called a medical cart is mainly described. A dashed line in FIG. 46 indicates the partial schematic shape of the medical cart. A dashed circle in FIG. 46 indicates a tire of the medical cart. The medical cart mainly runs in the left-right direction with the left direction in FIG. 46 as the front direction. The user puts cartridges into and takes the cartridges out of the charging apparatus 900 through an upper right portion in FIG. 46. To improve the workability of the insertion and removal of the cartridges, the entirety of the charging apparatus 900 is disposed inclined in a right rotational direction in FIG. 46. Hereinafter, the right side in FIG. 46 is referred to as a “front side” of the charging apparatus 900, and the left side in FIG. 46 is referred to as a “rear side” of the charging apparatus 900. An X-direction in FIG. 45 is a width direction. A Y-direction in FIG. 45 is a thickness direction. A Z-direction in FIG. 45 is a height or up-down direction.

A slot 731 is formed in the charging apparatus 900. The slot 731 is a recessed portion that accommodates cartridges according to the present embodiment. The inside of the slot 731 is composed of a bottom surface 732, a side surface 733, a side surface 734, a front surface 735 (one of main surfaces) on the front side, and a rear surface 736 (the other main surface) opposed to the front surface 735. On the front surface 735, a cutout 737 is formed. A height H from the lower end of the cutout 737 to the bottom surface 732 is smaller than the minimum height of the cartridges expected to be accommodated, thereby ensuring a space where the user grips the cartridges from above when the user puts in and takes out the cartridges.

In the present embodiment, the wireless power transmission unit 810 is provided on the front surface 735 side, and main surface pressing members 727 and springs 728 (not illustrated) are provided on the rear surface 736 side. As an example, four main surface pressing members 727 are provided.

Side surface pressing members 738 as a position adjustment unit for the cartridge 700 or the like protrude from the single side surface 733. Each of the side surface pressing members 738 is pivotable about one end of the side surface pressing member 738 and includes a twist coil spring (not illustrated) in a root portion of the side surface pressing member 738. The side surface pressing member 738 includes a roller 739 in an end portion on the opposite side and is biased by the twist coil spring in the direction in which the roller 739 separates from the side surface 733. When an external force is not applied to the side surface pressing member 738, a distance W from the roller 739 to the side surface 734 opposed to the side surface 733 is less than or equal to the minimum width of the cartridges expected to be accommodated. On the other hand, in a case where an external force is applied in the winding direction of the twist coil spring, the side surface pressing member 738 can be pivotally retracted to the position where the distance W is greater than the maximum width of the cartridges expected to be accommodated. The roller 739 is provided for the purpose of reducing the sliding contact resistance between the cartridges and the side surface pressing member 738 when the cartridges are inserted and removed. In addition to the provision of a roller, a ball chip may be used, or a material having low sliding contact resistance may be used. Although in the present embodiment, the side surface pressing members 738 are provided at two places along the height direction, similarly to the main surface pressing members 727, the number, the sizes, and the shapes of side surface pressing members 738 may be appropriately changed. A configuration may be employed in which the cartridges are pressed using a member having cushioning properties or elasticity, instead of being pressed by springs. Although in this case, side surface pressing members are provided only on a single side surface, side surface pressing members may be provided on both the side surfaces 733 and 734 and press the accommodated cartridges toward the center of the apparatus.

With reference to FIGS. 47A to 47C, the following describes behaviors when the cartridge 700 is accommodated in the charging apparatus 900.

As illustrated in FIG. 47A, when the user accommodates the cartridge 700 in the slot 731, the cartridge 700 abuts the side surface pressing members 738 and is guided to the side surface 734 side. Next, when the accommodated state proceeds to FIG. 47B, the cartridge 700 abuts the main surface pressing members 727 and is pressed to the front surface 735 side. When the cartridge 700 is eventually released from holding by the user and enters the state where the cartridge 700 is on the bottom surface 732, the position of the cartridge 700 is guided to the state of FIG. 47C where the cartridge 700 contacts the side surface 734 and the front surface 735, and is held. At this time, the bottom surface 732, the side surface 734, and the front surface 735 slide in contact with the cartridge 700, and therefore, it is desirable that these surfaces each be formed of a material having high sliding contact properties. Alternatively, sliding contact properties with the cartridge 700 may be improved by reducing a sliding contact area by providing a rib shape or a dot shape, or providing a rolling member such as a roller, a ball, or the like. Pressing the side surface 734 of the cartridge 700 prior to pressing the front surface 735 by the cartridge 700 is advantageous, as this allows a side surface holding force to be reduced. Thus, it is desirable that the side surface pressing members 738 and the main surface pressing members 727 have a positional relationship where the cartridge 700 abuts the side surface pressing members 738 before the main surface pressing members 727.

In the state of FIG. 47C, the wireless power reception unit 710 and the wireless power transmission unit 810 are disposed in the respective apparatuses in such a manner that the distance between the centers of the coils and the distance between the coils in the surface direction are as close to each other as possible. The cartridge 700 is held in the state of FIG. 47C, i.e., at the position where the wireless power reception unit 710 is pressed toward the wireless power transmission unit 810, whereby it is possible to maintain high wireless charging efficiency. Particularly, in a case where the charging apparatus 900 is mounted on a medical cart, the position of the cartridges in a slot is unstable due to vibration when the medical cart runs. A position holding unit according to the present embodiment is provided, whereby it is possible to improve the wireless charging efficiency even while the medical cart is running.

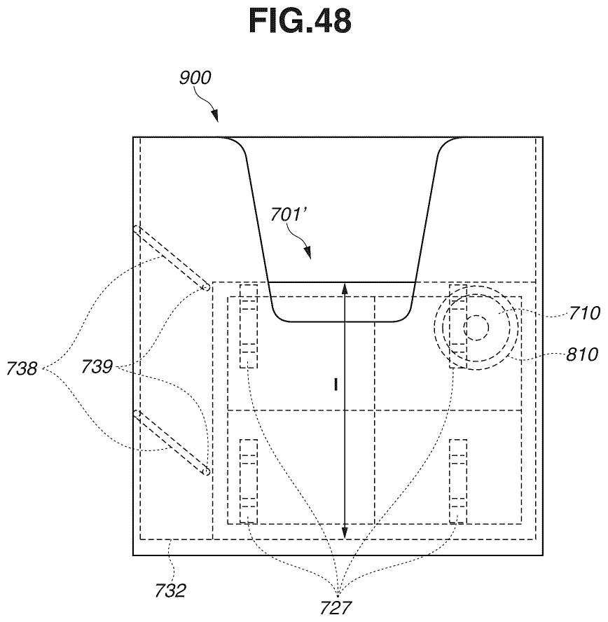

As described above, when the cartridge 700 is inserted into and removed from the charging apparatus 900, it is most natural that the user grips the thick portion 708b of the cartridge 700 as a series of operations from when imaging is performed. Thus, the present embodiment is based on the premise that the cartridge 700 is inserted into the slot 731 with the thick portion 708b above and with the back surface of the cartridge 700 on the front side so that a step between the thin portion 708a and the thick portion 708b is not caught on the entrance of the slot 731. On the other hand, a case is considered where a common wireless power transmission unit 810 transmits power when a cartridge 711′ having the smallest height (a height I) among the cartridges expected to be accommodated in the charging apparatus 900 is accommodated (FIG. 48) and when the cartridge 700 is accommodated. In this case, the wireless power transmission unit 810 needs to be disposed within the range of the height I from the bottom surface 732. To oppose the wireless power reception unit 710 in the cartridge 700 to the wireless power transmission unit 810 in such arrangement, the wireless power reception unit 710 is disposed in the thin portion 708a. As described above, a wireless charging unit is disposed on the assumption of cartridges having not only a variety of thicknesses but also a variety of widths or heights, whereby even a user owning a variety of cartridges can use a common charging apparatus. Also the manufacturers and providers of apparatuses can reduce the types of charging apparatuses to be prepared.

Although in the above description, a single wireless power reception unit 710 and a single wireless power transmission unit 810 transmit and receive power for charging, either one or both of the cartridge and the charging apparatus may include a plurality of wireless power reception units 710 and a single wireless power transmission unit 810, or include a single wireless power reception unit 710 and a plurality of wireless power transmission units 810, or include a plurality of wireless power reception units 710 and a plurality of wireless power transmission units 810. In this case, power is transmitted and received using any or all of the plurality of wireless power reception units 710 and wireless power transmission units 810. The position adjustment in the width or height direction of the cartridges can be omitted, and only the position adjustment in the thickness direction may be performed. For example, among the covers, some grid cover or the like is about 20 mm to 50 mm wider than a cartridge main body. Even if a cartridge with such a grid cover is accommodated in a charging apparatus in which only a single wireless power transmission unit is provided according to a cartridge without a cover, it is difficult to bring the distance between the center positions of coils close to 5 mm or less or the like. Even in such a case, with a plurality of coils, it is possible to ensure the wireless charging efficiency of wireless charging from any of the coils.

In addition to the above embodiments, a charging confirmation mechanism for confirming the charging efficiency of a cartridge may be provided in a charging apparatus. For example, the charging confirmation mechanism includes a detection unit, a confirmation unit, and a notification unit. The detection unit detects the presence or absence of the accommodation of the cartridge in the charging apparatus. In a case where the detection unit detects the accommodation of the cartridge, the confirmation unit confirms whether the cartridge reaches desired and defined charging efficiency when the cartridge is charged. Even in a case where the detection unit detects the accommodation of the cartridge, but the confirmation unit cannot confirm the desired charging efficiency, the notification unit issues a notification for urging the user to correct the accommodated state of cartridges. As the notification unit, a notification unit that performs predetermined sound generation, light emission, graphic display, or the like on the cartridge, the charging apparatus, or an external apparatus is possible. Such a charging confirmation mechanism is provided, whereby the user can perform charging work more certainly.

In any of the above embodiments, a position adjustment unit presses a cartridge in the direction in which a surface of the cartridge on which a wireless power reception unit is disposed is pressed toward a surface of a charging apparatus on which a wireless power transmission unit is disposed. Consequently, it is possible to deal with various cartridges. A single charging apparatus may charge any of a cartridge that changes its thickness, such as the cartridge 700, a cartridge having an approximately uniform thickness, such as the cartridge 711, a cartridge covered by a cover, and the like. In such various cartridges, a positional shift in a thickness direction and separation by a predetermined distance or more between a wireless power reception unit and a wireless power transmission unit is reduced, and the efficiency of wireless charging improves.

As described above, according to the present embodiment, even in a case where a cartridge varies such that, for example, the thickness of the cartridge is thin, thick, or both thin and thick, the stability of wireless charging in a charging apparatus is improved. Consequently, it is possible to use a common charging apparatus among a variety of cartridges. This saves the trouble of attaching and detaching an accessory to be used with a cartridge, such as a cover or the like, and the work efficiency for charging improves.

In the embodiments, a charging system (a power transmission/reception system) includes a charging apparatus and various cartridges that can be accommodated in the charging apparatus. Consequently, the charging apparatus can wirelessly charge the various cartridges stably and efficiently.

One of the radiation imaging apparatuses according to the ninth and tenth embodiments can also be applied to the radiation imaging apparatus 1601 of the radiation imaging system described with reference to FIG. 16 in the other embodiments.

While desired embodiments and modifications of the present disclosure have been described above, the present disclosure is not limited to these embodiments and modifications, and various changes and alterations may be made without departing from the scope and spirit of the disclosure. Furthermore, the above-described embodiments and modifications may be appropriately combined as needed.

The disclosures of the various embodiments and modifications include the following configurations.

### (Configuration 1)

A radiation imaging apparatus comprising:
a radiation detector configured to detect radiation;
a power supply unit configured to supply power to the radiation detector;
a housing configured to accommodate the radiation detector in a thin portion and accommodate the power supply unit in a thick portion; and
a power reception unit disposed in the thin portion and configured to receive supply of power from outside.

### (Configuration 2)

The radiation imaging apparatus according to configuration 1, wherein a panel portion of the radiation detector is disposed in the thin portion.

### (Configuration 3)

The radiation imaging apparatus according to configuration 1 or 2, wherein the power reception unit is a wireless power reception unit configured to wirelessly receive power from outside.

### (Configuration 4)

The radiation imaging apparatus according to configuration 1 or 2, wherein the power reception unit is a wired power reception unit configured to receive power via a wired connection from outside.

### (Configuration 5)

The radiation imaging apparatus according to configuration 3, wherein the wireless power reception unit includes a wireless coil and receives power with electromagnetic energy using an electromagnetic induction method.

### (Configuration 6)

The radiation imaging apparatus according to configuration 3 or 5, wherein the wireless power reception unit is disposed on a surface opposite to a radiation incident surface of a panel portion of the radiation detector in such a manner that at least a part of the wireless power reception unit overlaps the panel portion of the radiation detector in a plan view.

### (Configuration 7)

The radiation imaging apparatus according to configuration 5, wherein the wireless coil is arranged in such a manner that one end of the wireless coil is disposed in an end portion of the thick portion beyond an outer periphery of an effective imaging region of the radiation detector in a plan view.

### (Configuration 8)

The radiation imaging apparatus according to configuration 3, further comprising a first control unit configured to control the wireless power reception unit, wherein the first control unit is disposed in the thick portion.

### (Configuration 9)

The radiation imaging apparatus according to configuration 3, wherein a shield material configured to suppress radiation noise from the wireless power reception unit is disposed between a panel portion of the radiation detector and the wireless power reception unit.

### (Configuration 10)

The radiation imaging apparatus according to configuration 3, wherein a thermal insulating material configured to suppress temperature propagation due to heat generation in the wireless power reception unit is disposed between a panel portion of the radiation detector and the wireless power reception unit.

### (Configuration 11)

The radiation imaging apparatus according to configuration 3, further comprising a supporting base configured to support the radiation detector,
wherein an electromagnetic field transmitting material is disposed in a portion of the supporting base where the wireless power reception unit is disposed.

### (Configuration 12)

The radiation imaging apparatus according to configuration 3, wherein a wired power reception unit other than the wireless power reception unit is disposed in the thick portion.

### (Configuration 13)

The radiation imaging apparatus according to configuration 12, wherein the wired power reception unit is a power reception connector.

### (Configuration 14)

The radiation imaging apparatus according to configuration 12 or 13, further comprising:
a second control unit configured to control reception of power using the wireless power reception unit or the wired power reception unit,
wherein the second control unit switches between use of the wireless power reception unit and the wired power reception unit from one to the other, based on a power reception state of the wireless power reception unit or the wired power reception unit.

### (Configuration 15)

The radiation imaging apparatus according to configuration 4, wherein the wired power reception unit is a Universal Serial Bus (USB) connector.

### (Configuration 16)

The radiation imaging apparatus according to configuration 4, wherein the wired power reception unit is disposed on a surface opposite to a radiation incident surface of a panel portion of the radiation detector in such a manner that at least a part of the wired power reception unit overlaps the panel portion of the radiation detector in a plan view.

### (Configuration 17)

The radiation imaging apparatus according to configuration 4, wherein the wired power reception unit is disposed at a position that does not overlap a panel portion of the radiation detector in a plan view.

### (Configuration 18)

The radiation imaging apparatus according to configuration 1, further comprising:
a notification unit configured to issue a notification of predetermined information; and
a temperature sensor disposed in proximity of the power reception unit,
wherein in a case where a temperature detected by the temperature sensor is greater than or equal to a threshold and power received by the power reception unit is being used for an operation of the radiation imaging apparatus, the notification unit issues a notification of cautionary information.

### (Configuration 19)

The radiation imaging apparatus according to configuration 1, further comprising:
a notification unit configured to issue a notification of predetermined information; and
a temperature sensor disposed in proximity of the power reception unit,
wherein in a case where power received by the power reception unit is not being used for an operation of the radiation imaging apparatus, the notification unit notifies a power transmission side of power transmission stop information.

### (Configuration 20)

The radiation imaging apparatus according to configuration 1, further comprising:
a measurement unit configured to measure a time elapsed during reception of power or a time elapsed from an end of the reception of power; and
a notification unit configured to issue a notification of predetermined information,
wherein in a case where the radiation imaging apparatus is receiving power and the time elapsed during the reception of power is greater than or equal to a threshold, the notification unit issues a notification of cautionary information.

### (Configuration 21)

The radiation imaging apparatus according to configuration 1, further comprising:
a measurement unit configured to measure a time elapsed during reception of power or a time elapsed from an end of the reception of power; and
a notification unit configured to issue a notification of predetermined information,
wherein in a case where the radiation imaging apparatus is not receiving power and the time elapsed during the reception of power is less than a threshold, the notification unit issues a notification of cautionary information.

### (Configuration 22)

A radiation imaging apparatus comprising:
a radiation detector configured to detect radiation;
a power supply unit configured to supply power to the radiation detector;
a housing configured to accommodate the radiation detector in a thin portion and accommodate the power supply unit in a thick portion; and
a power reception unit disposed in the thick portion and configured to receive supply of power from outside.

### (Configuration 23)

The radiation imaging apparatus according to configuration 22, wherein the thick portion is thicker than the thin portion on an irradiation surface of radiation, and a recessed gripping portion is disposed on a back surface opposite to the irradiation surface.

### (Configuration 24)

The radiation imaging apparatus according to configuration 22 or 23, wherein the power reception unit is disposed in a sloping portion between the thick portion and the thin portion on an irradiation surface of radiation.

### (Configuration 25)

The radiation imaging apparatus according to configuration 22 or 23, wherein in a plan view, a width in a longitudinal direction of the thick portion is narrower than a width in the longitudinal direction of the thin portion.

### (Configuration 26)

The radiation imaging apparatus according to configuration 25, wherein in a plan view, the power reception unit is disposed on an inner side of a virtual straight line connecting an apex of the thick portion and an apex of the thin portion in proximity of the apex of the thick portion.

### (Configuration 27)

The radiation imaging apparatus according to configuration 25 or 26, wherein the power reception unit is disposed on a side surface in a short direction of the thick portion.

### (Configuration 28)

The radiation imaging apparatus according to configuration 27, wherein the power reception unit is disposed in a region in a range of 15 mm to 16 mm from a back surface opposite to an irradiation surface of radiation to the irradiation surface.

### (Configuration 29)

The radiation imaging apparatus according to any one of configurations 22 to 28, wherein the power reception unit is a power reception connector.

### (Configuration 30)

The radiation imaging apparatus according to any one of configurations 22 to 28, wherein the power reception unit is a wireless power reception unit configured to wirelessly receive and feed power from and to outside.

### (Configuration 31)

The radiation imaging apparatus according to configuration 22,
wherein a depression is formed in an end portion of a back surface opposite to an irradiation surface of radiation of the thick portion,
wherein a step between the depression and the back surface is provided in an outermost shape part of the depression, and
wherein the power reception unit is disposed on a side wall surface of the depression.

### (Configuration 32)

The radiation imaging apparatus according to configuration 31, wherein the power reception unit is disposed on an inner side of a virtual straight line connecting an apex on an outermost surface of the step and an apex of the side wall surface.

### (Configuration 33)

An accommodation member that accommodates a radiation imaging apparatus therein, the radiation imaging apparatus including a radiation detector for detecting radiation,
wherein a space in an accommodation unit for accommodating the radiation imaging apparatus includes at least a first region and a second region narrower than the first region, and
wherein the first region includes a power feeding unit configured to receive and feed power from and to the radiation imaging apparatus.

### (Configuration 34)

The accommodation member according to configuration 33, wherein the power feeding unit is a power feeding connector.

### (Configuration 35)

The accommodation member according to configuration 33, wherein the power feeding unit is a wireless power feeding unit configured to wirelessly receive and feed power from and to the radiation imaging apparatus.

### (Configuration 36)

The accommodation member according to any one of configurations 33 to 35, wherein the first region includes a supporting portion configured to support the radiation imaging apparatus.

### (Configuration 37)

The accommodation member according to any one of configurations 33 to 36, wherein the space in the accommodation unit includes a third region narrower than the first region and wider than the second region.

### (Configuration 38)

The accommodation member according to any one of configurations 33 to 37, wherein the accommodation member is provided in any of a medical cart, a power feeding stand, and a bucky stand.

### (Configuration 39)

A power reception/feeding system where a radiation imaging apparatus is accommodated in an accommodation member and the radiation imaging apparatus and the accommodation member receive and feed power from and to each other,
wherein the radiation imaging apparatus includes:
   a radiation detector configured to detect radiation;
   a power supply unit configured to supply power to the radiation detector;
   a housing configured to accommodate the radiation detector in a thin portion and accommodate the power supply unit in a thick portion; and
   a power reception unit disposed in the thick portion and configured to receive supply of power from outside, and
wherein in the accommodation member,
   a space in an accommodation unit for accommodating the radiation imaging apparatus includes at least a first region and a second region narrower than the first region, and
   the first region includes a power feeding unit configured to receive and feed power from and to the radiation imaging apparatus.

### (Configuration 40)

A wireless power feeding apparatus that wirelessly feeds power to a radiation imaging apparatus including a wireless power reception unit for receiving wireless feeding of power, the wireless power feeding apparatus comprising:
an accommodation unit configured to accommodate the radiation imaging apparatus;
a wireless power transmission unit configured to wirelessly feed power to the wireless power reception unit; and
an adjustment mechanism configured to, in a case where the radiation imaging apparatus is accommodated in the accommodation unit, adjust a position of the radiation imaging apparatus in such a manner that the wireless power transmission unit and the wireless power reception unit have a positional relationship where the wireless power feeding apparatus is able to wirelessly feed power to the radiation imaging apparatus.

### (Configuration 41)

The wireless power feeding apparatus according to configuration 40, wherein the wireless power reception unit is a wireless power reception method for receiving power for charging, and the wireless power transmission unit is a wireless power transmission method configured to transmit power for charging.

### (Configuration 42)

The wireless power feeding apparatus according to configuration 40 or 41, wherein the radiation imaging apparatus includes a radiation detection panel configured to convert radiation transmitted through an object into an electric signal.

### (Configuration 43)

The wireless power feeding apparatus according to any one of configurations 40 to 42,
wherein the adjustment mechanism includes a main surface pressing member, and
wherein the main surface pressing member presses a main surface of the accommodated radiation imaging apparatus in a thickness direction in such a manner that the wireless power reception unit comes close to the wireless power transmission unit.

### (Configuration 44)

The wireless power feeding apparatus according to any one of configurations 40 to 43,
wherein the adjustment mechanism includes a side surface pressing member, and
wherein the side surface pressing member presses a side surface of the accommodated radiation imaging apparatus in a direction intersecting a thickness direction in such a manner that the wireless power reception unit comes close to the wireless power transmission unit.

### (Configuration 45)

The wireless power feeding apparatus according to any one of configurations 40 to 44,
wherein the adjustment mechanism includes an inclined roller member, and
wherein the inclined roller member is disposed at a place that where a-weight of the accommodated radiation imaging apparatus is applied, and the inclined roller member adjusts the position of the accommodated radiation imaging apparatus by rotation of the inclined roller member in such a manner that the wireless power reception unit comes close to the wireless power transmission unit.

### (Configuration 46)

The wireless power feeding apparatus according to any one of configurations 40 to 45, further comprising an accommodation unit including a slot in which the radiation imaging apparatus is accommodated.

### (Configuration 47)

The wireless power feeding apparatus according to configuration 46, wherein in the accommodation unit, among surfaces forming the slot, at least one surface that receives application of the weight of the accommodated radiation imaging apparatus is inclined from horizontal or vertical.

### (Configuration 48)

The wireless power feeding apparatus according to configuration 47, wherein a bottom surface forming the slot is inclined from horizontal in such a manner that one side in a short direction of the slot is lower than the other side facing the one side in the short direction.

### (Configuration 49)

The wireless power feeding apparatus according to configuration 47, wherein a bottom surface and at least one of main surfaces forming the slot are inclined from horizontal and vertical in such a manner that one side in a longitudinal direction of the slot is lower than the other side facing the one side in the longitudinal direction, while the bottom surface and the at least one main surface keep perpendicular to each other.

### (Configuration 50)

The wireless power feeding apparatus according to any one of configurations 40 to 45, further comprising an accommodation unit including a slot in which the radiation imaging apparatus is accommodated,
wherein the wireless power reception unit is disposed on a first main surface of the radiation imaging apparatus,
wherein the wireless power transmission unit is disposed on a second main surface of the slot,
wherein in a case where the radiation imaging apparatus is accommodated in the accommodation unit, the radiation imaging apparatus moves in friction contact with a bottom surface of the slot by application of a weight of the radiation imaging apparatus, and the wireless power reception unit faces the wireless power transmission unit, and
wherein the adjustment mechanism adjusts a position of the radiation imaging apparatus to press the first main surface toward the second main surface in a state where the wireless power reception unit and the wireless power transmission unit face each other.

### (Configuration 51)

The wireless power feeding apparatus according to any one of configurations 40 to 45, further comprising an accommodation unit including a slot in which the radiation imaging apparatus is accommodated,
wherein the wireless power reception unit is disposed on a first main surface of the radiation imaging apparatus,
wherein the wireless power transmission unit is disposed on a second main surface of the slot,
wherein in a case where the radiation imaging apparatus is accommodated in the accommodation unit, the wireless power reception unit faces the wireless power transmission unit, and
wherein the adjustment mechanism adjusts a position of the radiation imaging apparatus to press the first main surface toward the second main surface in a state where the wireless power reception unit and the wireless power transmission unit face each other.

### (Configuration 52)

The wireless power feeding apparatus according to configuration 51, wherein the first main surface is a surface on a back side when viewed in a thickness direction of the radiation imaging apparatus among main surfaces of the radiation imaging apparatus.

### (Configuration 53)

The wireless power feeding apparatus according to any one of configuration 50 to 52, wherein the adjustment mechanism presses the first main surface toward the second main surface in such a manner that a facing distance in a thickness direction of the radiation imaging apparatus between the wireless power reception unit and the wireless power transmission unit is 5 mm or less.

### (Configuration 54)

The wireless power feeding apparatus according to any one of configurations 40 to 53,
wherein the wireless power feeding apparatus accommodates the radiation imaging apparatus having a thickness and/or width different from the radiation imaging apparatus of another, and
wherein the adjustment mechanism brings the wireless power reception unit and the wireless power transmission unit close to each other, regardless of a thickness and a width of the radiation imaging apparatus.

### (Configuration 55)

A power transmission/reception system comprising:
a radiation imaging apparatus including a wireless power reception unit configured to receive wireless feeding of power; and
a wireless power feeding apparatus configured to accommodate the radiation imaging apparatus,
wherein the wireless power feeding apparatus includes:
   a wireless power transmission unit configured to wirelessly feed power to the wireless power reception unit; and
   an adjustment mechanism configured to adjust, in a case where the radiation imaging apparatus is accommodated, a position of the radiation imaging apparatus in such a manner that the wireless power transmission unit and the wireless power reception unit have a positional relationship where the wireless power feeding apparatus is able to wirelessly feed power to the radiation imaging apparatus.

### (Configuration 56)

The power transmission/reception system according to configuration 55, wherein
the radiation imaging apparatus includes:
   a thin portion partially or entirely overlapping an effective imaging region when viewed in a thickness direction of the radiation imaging apparatus; and
   a thick portion thicker than the thin portion.

### (Configuration 57)

The power transmission/reception system according to configuration 56,
wherein the wireless power reception unit is disposed in the thin portion, and
wherein a control unit configured to control the wireless power reception unit is disposed in the thick portion.

### (Configuration 58)

The power transmission/reception system according to any one of configurations 55 to 57,
wherein a cover is attached to the radiation imaging apparatus, and
wherein the adjustment mechanism brings the wireless power reception unit and the wireless power transmission unit close to each other, regardless of presence or absence of the attachment of the cover to the radiation imaging apparatus.

### (Configuration 59)

The power transmission/reception system according to configuration 58,
wherein the cover is at least one type selected from:
   a grid cover configured to reduce predetermined radiation;
   an imaging assistance cover configured to adjust a contact state between the radiation imaging apparatus and an object; and
   a protective cover configured to protect the radiation imaging apparatus from contamination and water immersion.

### (Configuration 60)

The power transmission/reception system according to any one of configurations 55 to 59, wherein one or both of the radiation imaging apparatus and the wireless power feeding apparatus include a plurality of one or both of the wireless power reception units and the wireless power transmission units.

### (Configuration 61)

A radiation imaging system comprising:
a radiation generating apparatus configured to emit radiation to an object;
the radiation imaging apparatus according to any one of configurations 1 to 32; and
a calculation processing apparatus configured to perform a predetermined calculation process based on information acquired by the radiation imaging apparatus.

The present disclosure is not limited to the above-described embodiments, and various changes and modifications may be made without departing from the spirit and scope of the disclosure. Accordingly, the following claims are appended in order to publicly define the scope of the disclosure.

According to the present disclosure, it is possible to obtain a radiation imaging apparatus that achieves a suitable configuration and suitable arrangement of a power reception unit that suits a case where the thickness of a radiation detector is thinned.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A radiation imaging apparatus comprising:
a radiation detector configured to detect radiation;
a power supply unit configured to supply power to the radiation detector;
a housing configured to accommodate the radiation detector in a thin portion and accommodate the power supply unit in a thick portion; and
a power reception unit disposed in the thin portion and configured to receive supply of power from outside.

2. The radiation imaging apparatus according to claim 1, wherein a panel portion of the radiation detector is disposed in the thin portion.

3. The radiation imaging apparatus according to claim 1, wherein the power reception unit is a wireless power reception unit configured to wirelessly receive power from outside.

4. The radiation imaging apparatus according to claim 1, wherein the power reception unit is a wired power reception unit configured to receive power via a wired connection from outside.

5. The radiation imaging apparatus according to claim 3, wherein the wireless power reception unit includes a wireless coil and receives power with electromagnetic energy using an electromagnetic induction method.

6. The radiation imaging apparatus according to claim 3, wherein the wireless power reception unit is disposed on a surface opposite to a radiation incident surface of a panel portion of the radiation detector in such a manner that at least a part of the wireless power reception unit overlaps the panel portion of the radiation detector in a plan view.

7. The radiation imaging apparatus according to claim 5, wherein the wireless coil is arranged in such a manner that one end of the wireless coil is disposed in an end portion of the thick portion beyond an outer periphery of an effective imaging region of the radiation detector in a plan view.

8. The radiation imaging apparatus according to claim 3, further comprising a first control unit configured to control the wireless power reception unit,
wherein the first control unit is disposed in the thick portion.

9. The radiation imaging apparatus according to claim 3, wherein a shield material configured to suppress radiation noise from the wireless power reception unit is disposed between a panel portion of the radiation detector and the wireless power reception unit.

10. The radiation imaging apparatus according to claim 3, wherein a thermal insulating material configured to suppress temperature propagation due to heat generation in the wireless power reception unit is disposed between a panel portion of the radiation detector and the wireless power reception unit.

11. The radiation imaging apparatus according to claim 3, further comprising a supporting base configured to support the radiation detector,
wherein an electromagnetic field transmitting material is disposed in a portion of the supporting base where the wireless power reception unit is disposed.

12. The radiation imaging apparatus according to claim 3, wherein a wired power reception unit other than the wireless power reception unit is disposed in the thick portion.

13. The radiation imaging apparatus according to claim 12, wherein the wired power reception unit is a power reception connector.

14. The radiation imaging apparatus according to claim 12, further comprising:
a second control unit configured to control reception of power using the wireless power reception unit or the wired power reception unit,
wherein the second control unit switches between use of the wireless power reception unit and the wired power reception unit from one to the other, based on a power reception state of the wireless power reception unit or the wired power reception unit.

15. The radiation imaging apparatus according to claim 4, wherein the wired power reception unit is a Universal Serial Bus (USB) connector.

16. The radiation imaging apparatus according to claim 4, wherein the wired power reception unit is disposed on a surface opposite to a radiation incident surface of a panel portion of the radiation detector in such a manner that at least a part of the wired power reception unit overlaps the panel portion of the radiation detector in a plan view.

17. The radiation imaging apparatus according to claim 4, wherein the wired power reception unit is disposed at a position that does not overlap a panel portion of the radiation detector in a plan view.

18. The radiation imaging apparatus according to claim 1, further comprising:
a notification unit configured to issue a notification of predetermined information; and
a temperature sensor disposed in proximity of the power reception unit,
wherein in a case where a temperature detected by the temperature sensor is greater than or equal to a threshold and power received by the power reception unit is being used for an operation of the radiation imaging apparatus, the notification unit issues a notification of cautionary information.

19. The radiation imaging apparatus according to claim 1, further comprising:
a notification unit configured to issue a notification of predetermined information; and
a temperature sensor disposed in proximity of the power reception unit,
wherein in a case where power received by the power reception unit is not being used for an operation of the radiation imaging apparatus, the notification unit notifies a power transmission side of power transmission stop information.

20. The radiation imaging apparatus according to claim 1, further comprising:
a measurement unit configured to measure a time elapsed during reception of power or a time elapsed from an end of the reception of power; and
a notification unit configured to issue a notification of predetermined information,
wherein in a case where the radiation imaging apparatus is receiving power and the time elapsed during the reception of power is greater than or equal to a threshold, the notification unit issues a notification of cautionary information.

21. The radiation imaging apparatus according to claim 1, further comprising:
a measurement unit configured to measure a time elapsed during reception of power or a time elapsed from an end of the reception of power; and
a notification unit configured to issue a notification of predetermined information,
wherein in a case where the radiation imaging apparatus is not receiving power and the time elapsed during the reception of power is less than a threshold, the notification unit issues a notification of cautionary information.

22. A radiation imaging apparatus comprising:
a radiation detector configured to detect radiation;
a power supply unit configured to supply power to the radiation detector;
a housing configured to accommodate the radiation detector in a thin portion and accommodate the power supply unit in a thick portion; and
a power reception unit disposed in the thick portion and configured to receive supply of power from outside,
wherein the power reception unit is any of a sloping surface power reception unit disposed in proximity of a sloping surface linking the thick portion and the thin portion, a side surface power reception unit disposed on a side surface that does not come into contact with a flat floor surface among side surfaces of the thick portion, and a wireless power reception unit configured to receive wireless feeding of power.

23. A wireless power feeding apparatus that wirelessly feeds power to a radiation imaging apparatus including a wireless power reception unit for receiving wireless feeding of power, the wireless power feeding apparatus comprising:
an accommodation unit configured to accommodate the radiation imaging apparatus;
a wireless power transmission unit configured to wirelessly feed power to the wireless power reception unit; and
an adjustment mechanism configured to passively operate in a case where the radiation imaging apparatus is accommodated in the accommodation unit, and adjust a position of the radiation imaging apparatus in such a manner that the wireless power transmission unit and the wireless power reception unit have a positional relationship where the wireless power feeding apparatus is able to wirelessly feed power to the radiation imaging apparatus.

24. A radiation imaging system comprising:
a radiation generating apparatus configured to emit radiation to an object;
the radiation imaging apparatus according to claim 1; and
a calculation processing apparatus configured to perform a predetermined calculation process based on information acquired by the radiation imaging apparatus.
